(19) United States
(12) Patent Application Publication
(10) Pub. No.: US 2025/0361572 A1
(43) Pub. Date: Nov. 27, 2025

(54) MULTIPLEXED PATHOGEN DETECTION USING NANOPLASMONIC SENSOR FOR HUMAN PAPILLOMAVIRUS

(71) Applicant: Nanopath Inc., Cambridge, MA (US)

(72) Inventors: Alison Burklund, Cambridge, MA (US); Amogha Tadimety, Cambridge, MA (US); M. Nabuan Naufer, Billerica, MA (US)

(21) Appl. No.: 18/875,074

(22) PCT Filed: Jun. 15, 2023
(86) PCT No.: PCT/US2023/068549

§ 371 Date: Dec. 13, 2024

Related U.S. Application Data
(60) Provisional application No. 63/352,985, filed on Jun. 16, 2022.

(51) Int. Cl. C12Q 1/70; C12Q 1/6837

(52) U.S. Cl. CPC C12Q 1/708; C12Q 1/6837

(57) ABSTRACT

Disclosed herein includes a nanoplasmonic sensor for molecular characterization of human papillomavirus (HPV), and diseases and disorders associated with HPV infection. In some embodiments. the nanoplasmonic sensor can also be used at the point-of-care. The nanoplasmonic sensor utilizes an optical phenomenon that occurs between a metal nanoparticle and a dielectric—localized surface plasmon resonance (LSPR)—for the detection of viral nucleic acids. In some embodiments. the spectral peak shift is a function of target sequence concentration.

### INCORPORATION BY REFERENCE TO ANY PRIORITY APPLICATIONS

This application claims priority to U.S. Provisional Patent Application No. 63/352,985 filed on Jun. 16, 2022. Any and all applications, if any, for which a foreign or domestic priority claim are hereby incorporated by reference in their entireties.

### REFERENCE TO SEQUENCE LISTING

The present application is being filed along with a Sequence Listing submitted electronically in XML format. The Sequence Listing is provided as a file entitled NPATH.008WOSEQLISTING.xml, created Jun. 15, 2023, which is approximately 8,116 bytes in size. The information in the electronic format of the Sequence Listing is incorporated herein by reference in its entirety.

### BACKGROUND

### Field of the Invention

This disclosure is related to the field of molecular detection. Specifically, the disclosure describes a method for functionalization of nanoplasmonic sensor and a functionalized nanoplasmonic sensor for the molecular characterization of the human papillomavirus (HPV) and subsequent diseases and disorder related to HPV infection in a subject.

### Description of the Related Art

Cervical cancer is the fourth most frequent cancer in women and represents 7.5% of all female cancer deaths. Over 80% of worldwide cervical cancer cases occur in low- and middle-income countries, and almost all of these cases are caused by human papillomavirus (HPV). Hundreds of HPV genotypes have been identified. Approximately 40 HPV genotypes affect the genital tract and are classified as either high-risk or low-risk. The current gold standard diagnostic is a pap smear, which requires a cervical swab and analysis from an experienced cytologist. In developed countries, there are screening programs that identify and treat pre-cancerous lesions early, preventing up to 80% of cervical cancers. In contrast, in low-and middle-income countries only 5% of women receive pap smear testing each year, and some wait up to 6 months for a result. False information, cultural sensitivities, concerns from partners, and stigmatization of women exacerbate testing issues. These discrepancies in testing availability are thought to be main contributors to the higher cervical cancer rates and mortality rates observed in lower income countries. By bringing faster, less invasive screening tests to resource limited settings, treatment rates could significantly increase, improving patient outcomes in these areas.

Many existing and emerging diagnostic platforms employ polymerase chain reaction (PCR) for detection of genotype-specific HPV DNA in a cervical swab sample. On average, these tests have a higher sensitivity and specificity than other widely utilized techniques, such as pap smear or visual inspection with acetic acid, but are often too complicated for routine screening in low-resource settings. If a patient could be stratified as high-risk at the point-of-care using a low-cost HPV genotyping technology, pre-cancers and early-stage cervical cancers can be treated in a single office visit.

### SUMMARY OF THE INVENTION

Disclosed herein is a nanoplasmonic sensor. In some embodiments, the nanoplasmonic sensor comprises: an array of functionalized sensors, wherein each of the functionalized sensors in the array comprises an array of nanostructures conjugated to a biological probe, and the biological probe is configured to detect the presence of a human papillomavirus. In some embodiments, at least one of the functionalized sensors in the array comprises a different biological probe for detecting a different segment of a human papillomavirus from the other functionalized sensors. In some embodiments, the nanoplasmonic sensor is configured to simultaneously detect multiple strands, segments, particles, mutants, and/or species of the human papillomaviruses. In some embodiments, each of the functionalized sensors in the array comprises a different biological probe. In some embodiments, the human papillomavirus is selected from the group consisting of HPV18, HPV16, hrHPV, HPV type 16, HPV type 18, HPV type 31, HPV type 33, HPV type 35, HPV type 39, HPV type 45, HPV type 51, HPV type 52, HPV type 56, HPV type 58, HPV type 59, HPV type 66, HPV type 68, and a derivative/mutant strain thereof. In some embodiments, the biological probe has a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In some embodiments, the nanostructures comprise gold. In some embodiments, the nanostructures in the array are regularly-spaced apart with a spacing of from about 100 nm and about 1000 nm, and each nanostructure has a square shape with a side dimension of from about 50 nm to about 400 nm. In some embodiments, the nanostructures have a thickness of from about 20 nm to about 75 nm.

Also disclosed herein is a method for detecting the presence of one or more human papillomaviruses. In some embodiments, the method comprises: (1) exposing the nanoplasmonic sensor of any of the embodiments disclosed herein to a bodily fluid sample of a patient suspecting of having an human papillomavirus infection, (2) illuminating a light at a series of wavelengths onto each of the functionalized sensors, and (3) collecting absorbance, transmittance, or extinction data of each functionalized sensor. In some embodiments, the method further comprises comparing the collected absorbance, transmittance, or extinction data of each functionalized sensor with a baseline data of each of the functionalized sensor prior to exposure to the bodily fluid sample. In some embodiments, the comparing step reveals an optical peak shift when a human papillomavirus is detected. In some embodiments, the amount of the optical peak shift is correlated to the concentration of the human papillomavirus in the bodily fluid sample. In some embodiments, the bodily sample comprises urine. In some embodiments, at least one of the functionalized sensors in the array comprises a different biological probe for detecting a different segment of a human papillomavirus from the other functionalized sensors. In some embodiments, the human papillomavirus is selected from the group consisting of HPV18, HPV16, hrHPV, HPV type 16, HPV type 18, HPV type 31, HPV type 33, HPV type 35, HPV type 39, HPV type 45, HPV type 51, HPV type 52, HPV type 56, HPV type 58, HPV type 59, HPV type 66, HPV type 68, and a derivative/mutant strain thereof. In some embodiments, the biological probe independently has a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. In some embodiments, each of the functionalized sensors in the array comprises a different biological probe. In some embodiments, multiple strands, segments, particles, mutants, and/or species of human papillomaviruses are detected simultaneously. In some embodiments, the method is configured to be performed at the point of care.

Another method for detecting the presence of one or more human papillomavaruus comprises providing a sensor comprising one or more biological probes designed to detect target nucleic acid sequences derived from one or more human papillomaviruses, exposing the sensor to a sample that is suspected to contain one or more human papillomaviruses, and collecting electrical, fluorescent, absorbance, transmittance, and/or extinction data from the sensor. In some embodiments, the one or more biological probes were selected using computational and/or bioinformatic methods. In some embodiments, the one or more biological probes contain intentionally varying degrees of mismatch with the target nucleic acids. In some embodiments, the one or more biological probes are designed to bind multiple target nucleic acid sequences. In some embodiments, one of the biological probes can bind nucleic acids derived from more than one human papillomaviruses. In some embodiments, the one or more biological probes are designed to bind nucleic acid sequences specific to one high-risk HPV genotypes.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A depict one embodiment of a plasmonic-resonance sensing device.

FIG. 1B depicts one embodiment of an array of nanostructures in a sensor of the plasmonic-resonance sensing device.

FIGS. 2A-2B depict non-limiting example schematics of selected geometries and fabrication maps. FIG. 1A illustrates a schematic of a grid with labeled dimensions for length, width, thickness, and periodicity of nanostructures. FIG. 1B illustrates a schematic of a map of arrangement of dimensions for dose matrix test.

FIG. 3 shows extinction curves of a non-limiting example of regular gold nanorod array at three bulk refractive indices.

FIGS. 4A-4B depict examples of PNA-DNA Binding Simulations. The simulations are of conformal layers representing PNA and DNA binding to gold nanostructure. The two geometries demonstrated here are (FIG. 3A) repeating nanorod array (130 nm×40 nm) and (FIG. 3B) repeating nanosquare array (95 nm×95 nm).

FIG. 5A shows the extinction curve for bulk refractive index sensitivity simulations in one embodiment of the nanostructure arrays at three refractive indexes. FIG. 5B depicts the refractive index sensitivities of uncoupled nanorods and the nanostructure array.

FIG. 6A shows the extinction curve for bulk refractive index sensitivity simulations in one embodiment of the nanostructure arrays at three refractive indexes. FIG. 6B depicts the refractive index sensitivities of uncoupled nanorods and the nanostructure array.

FIG. 7A shows the extinction curve for bulk refractive index sensitivity simulations in one embodiment of the nanostructure arrays at three refractive indexes. FIG. 7B depicts the refractive index sensitivities of uncoupled nanorods and the nanostructure array.

FIG. 8 depicts the experimental transmission spectra for 5 different nanoarray geometries.

FIG. 9 depicts the simulated transmission spectra for each 5 different nanoarray geometries.

FIG. 10 depicts a CAD drawing of post array polymer well mold and fabricated well, with coordinates aligned over the sensor array.

FIGS. 11A-11B depict two alternative views of a 3D printed mold for a fabricated polymer well.

FIGS. 11C-11D depict two alternative views of the final fabricated well array, made from the mold of FIGS. 11A and 11B.

FIGS. 11E-11I depict additional embodiments of micro-well fixtures.

FIGS. 12A-C depict one embodiment of the automatic pipette system. FIG. 12A depicts the overall system, with pipette holder on the left, tip box, 96-well plate holder, and custom chip adapter. FIG. 12B depicts the tip box aligned under pipette holder. FIG. 12C depicts the 96 well plate and adapter during functionalization.

FIG. 13 depicts one embodiment of inclusivity/cross-reactivity of HPV probes. In the “inclusivity section,” darker boxes denote highly probable specific binding whereas light boxes predicts thermodynamically less stable hybridization with hrHPV. Similarly, in the “X-reactivity” section, dark and light shades predict high and low probable hybridization with non-specific HPV types, respectively. The degree of binding-affinities are based on thermodynamic predictions of duplex stability governed by nearest neighbor interactions.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

A plasmon-resonance sensing device employing ordered array nanostructure ensembles is described herein. The ordered array of nanostructures allows for coupling to diffractive photonic modes, which can be used to improve sensor sensitivity. The nanostructure dimension and geometry are tailored to provide high quality signal and large optical shifts upon modeled analyte binding.

Disclosed herein is a nanoplasmonic biosensor for point-of-care molecular characterization of human papillomavirus. Some embodiments relate to a novel nanoplasmonic sensor fabrication methodology for rapid (<15 min) and highly specific genotyping of human papilloma viruses (HPVs). The nanoplasmonic sensor of the present application utilizes an extremely sensitive optical transduction methods based upon light-matter interactions at the nanoscale, enabling detection of target nucleic acid via hybridization events occurring at the sensor's surface. The technology of the present disclosure employs an optical phenomenon that occurs between a metal nanoparticle and a dielectric—localized surface plasmon resonance (LSPR)—for the detection of viral nucleic acids. LSPR is observed when the wavelength of incident light is larger than the size of the conductive nanoparticles and presents an opportunity for highly sensitive detection of specific nucleic acid sequences. In this work. gold nanoarrays are covalently functionalized with biological probes. The nanoparticles result in highly confined electric fields of LSPR modes, which serve as a sensitive transducer to changes in the local dielectric environment (i.e., a binding event). Upon hybridization to the nucleic acid target sequences, there are successive red shifts in the spectral peak as a function of target sequence concentration.

The panel of any of the present embodiments was designed to enable genotyping of HPV16 and other high-risk HPVs. A negative control was also included on the panel for specificity. In a patient cohort of 50 patients from 3 clinical sites (in 3 countries), there was >92% sensitivity and 100% specificity for single-genotype identification (HPV16) and pooled high-risk HPV genotyping from processed cervical swabs. Through careful in silico analysis, a new panel of rationally designed peptide nucleic acid (PNA) probes were also created, including a group of five consensus probes with high inclusivity and minimal cross-reactivity to other low-risk HPVs. These results suggest a broadly applicable nanosensor fabrication method, as well as a compelling initial platform for HPV genotyping.

### Plasmon-Resonance Sensing Devices

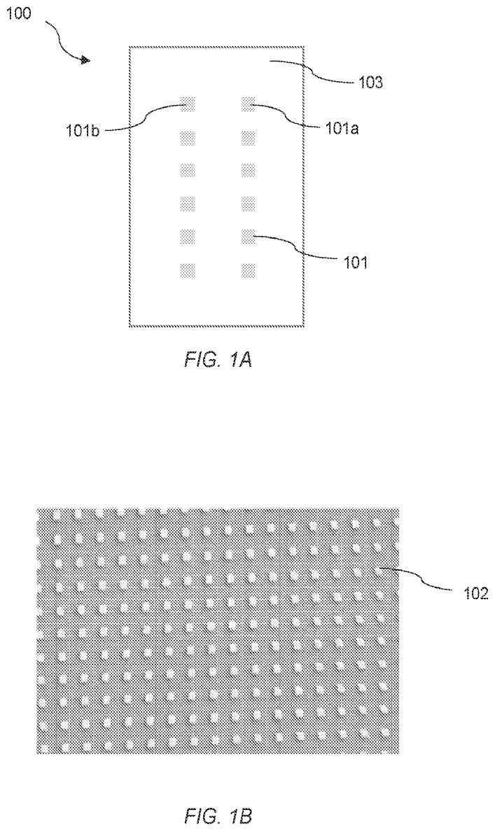

Disclosed herein is a plasmon-resonance sensing device. As shown in FIG. 1A and 1B, the plasmon-resonance sensing device 100 comprises an array of sensors 101. Each sensor 101 comprises an array of nanostructures 102 that are regularly spaced apart. In some embodiments, the nanostructures 102 are regularly spaced apart with a spacing of about 100 nm, about 200 nm, about 300 nm, about 500 nm, about 750 nm, about 1000 nm, about 1200 nm, about 1500 nm, about 1800 nm, about 2000 nm, or any distance that is between about 100 nm and about 2000 nm, between the nanostructures. In some embodiments, the array of nanostructures are regularly spaced apart with a spacing of from about 100 nm to about 2000 nm, from about 100 nm to about 1800 nm, from about 100 nm to about 1600 nm, from about 100 nm to about 1400 nm, from about 100 nm to about 1200 nm, from about 100 nm to about 1000 nm. from about 200 nm to about 900 nm, from about 300 nm to about 800 nm. from about 100 nm to about 400 nm, from about 200 nm to about 500 nm, from about 300 nm to about 600 nm, from about 400 nm to about 700 nm. from about 500nm to about 800 nm, from about 600 nm to about 900 nm, from about 700 nm to about 1000 nm, from about 500 nm to about 2000 nm, or from about 500 nm to about 1500 nm between the nanostructures.

The nanostructures in the array may have various shapes. For example, the nanostructures may have a rectangular shape, a circular shape, a triangular shape, a star shape, a pentagon shape, a parallelogram shape. a diamond shape, or a square shape. Preferably, each of the nanostructures in the array has a square shape. In some embodiments, each nanostructure has a side dimension of about 50 nm, about 75 nm, about 100 nm, about 150 nm, about 200 nm, about 250 nm, about 300 nm, about 350 nm, or about 400 nm, or any integer that is between about 50 to about 400 nm. In some embodiments, the square shape has a side dimension of from about 50 nm to about 400 nm, from about 100 nm to about 350 nm, from 150 nm to about 300 nm, from about 50 nm to about 150 nm, from about 100 nm to about 200 nm, from 150 nm to about 250 nm, from about 200 nm to about 300 nm, from about 250 nm to about 350 nm, or from about 300 nm to about 400 nm, or any range that is between about 50 nm and about 400 nm.

In some embodiments, the nanostructures in the array may have a thickness of about 20 nm, about 25 nm, about 30 nm, about 35 nm, about 40 nm, about 45 nm, about 50 nm, about 60 nm, about 65 nm, about 70 nm. about 75 nm, or any integer between about 20 and about 75 nm. In some embodiments, the nanostructures in the array may have a thickness of from about 20 nm to about 75 nm, from about 25 nm to about 70 nm, from about 30 nm to about 65 nm, from about 35 nm to about 60 nm, from about 30 nm to about 55 nm, or any range that is between about 20 and about 75 nm.

The nanostructures comprise a metal. For example, the nanostructures may comprise gold, platinum, aluminum, silver, or copper. Preferably, the nanostructure comprises gold. In some embodiments, the nanostructures comprise a single metal. In some embodiments, the nanostructures comprise a mixture of metals.

In some embodiments, the nanostructures in the array are conjugated with a biological probe. The biological probe is configured to bind to an analyte. The binding of the analyte to the biological probe alters the surface properties of the nanostructure. thereby causing a change in localized surface plasmon resonance. In some embodiments, the biological probe comprises one or more of a protein. peptide strand, amino acid, RNA strand, DNA strand, or and/or nucleotide. In some embodiments, the biological probe comprises one or more of a modified protein, modified peptide, modified amino acid, modified RNA strand, modified DNA strand, and/or modified nucleotide. In some embodiments, the biological probe comprises at least one of: a peptide-nucleic acid, an aptamer, an antibody, an antibody fragment, a complimentary DNA, and/or an enzyme. In some embodiments, the biological probe is selected from the group consisting of a peptide-nucleic acid, an aptamer. an antibody, an antibody fragment, a complimentary DNA. and an enzyme.

In some embodiments, at least a first sensor 101a in the array of sensors comprises nanostructures 102 conjugated with a first biological probe. In some embodiments, at least a second sensor 101b in the array of sensors comprises nanostructures conjugated with a second biological probe. In some embodiments, at least a third sensor in the array of sensors comprises nanostructures conjugated with a third biological probe. In some embodiments, at least a fourth sensor in the array of sensors comprises nanostructures conjugated with a fourth biological probe. In some embodiments, at least a fifth sensor in the array of sensors comprises nanostructures conjugated with a fifth biological probe. In some embodiments, a “n” number of sensors in an array of sensors comprises nanostructures conjugated to an “n” number of biological probes, wherein “n” is any number from 1 to 2000 In some embodiments, 6 or 12 sensors may be presented in the array of sensors on a substrate 103. In some embodiments, the sensors may have an area of from about 1 μm² to about 1 mm². In some embodiments, the sensors may have an area of from about 10 μm² to about 1 mm², about 50 μm² to about 1 mm², about 100 μm² to about 1 mm², about 200 μm² to about 1 mm², about 400 μm² to about 1 mm², or about 500 μm² to about 1 mm².

The substrate 103 may be a dielectric or non-conductive substrate. In some embodiments, the substrate 103 is transparent to allow the sensors to be exposed to the incident light through the substrate 103. For example, the substrate 103 may be a glass, a plastic, or a polymeric substrate. In some embodiments, the substrate 103 may be a polymer substrate or a plastic substrate. The substrate and the sensor array on the substrate may be integrated with a microfluidic module to provide a means for introducing or exposing the sample to the sensors.

### Analyte Detection

Disclosed herein is a method for detecting an analyte in a sample. In some embodiments, the method comprises exposing at least one sensor 101 in the plasmon-resonance sensing device 100 of any of the embodiments disclosed herein to a sample. The sample may or may not comprise the target analyte. The plasmon-resonance sensing device 100 can be utilized to detect the presence of an analyte (i.e., a target analyte). In some embodiments, the method comprises exposing at least two sensors in the plasmon-resonance sensing device 100 of any of the embodiments disclosed herein to a sample. In some embodiments, the method comprises exposing at least three sensors, at least four sensors, at least 5 sensors, or at least 6 sensors in the plasmon-resonance sensing device 100 of any of the embodiments disclosed herein to a sample. In some embodiments, the method comprises exposing an “n” number of sensors in the plasmon-resonance sensing device of any of the embodiments disclosed herein to a sample, wherein “n” is any number from 1 to 2000. In some embodiments, the array of sensors is exposed to the sample. The sample may comprise a bodily fluid, such as blood, plasma, mucus, serum, urine, or saliva, etc. Mucus can be collected via cervical swabs, vaginal swabs, or nasal swabs. When the at least one sensor 101 is exposed to the sample. the biological probe in each sensor would selectively bind to the analyte that the biological probe is configured to bine.

Optionally, the at least one sensor may be subject to a heating step after the exposure to the sample. In some embodiments. the at least one sensor is heated up to about 85° C. or any temperature between 25° C. and 85° C. In some embodiments, the at least one sensor may be exposed to heat before, during, or after subsequent steps. In some embodiments, the at least one sensor may be exposed to heat before, during, or after the measurement.

The method for detecting or sensing an analyte further comprises illuminating a light onto the at least one sensor. In some embodiments, the method comprises illuminating a light at a series of wavelengths onto the at least one sensor. In some embodiments, the light may be emitted from a light source in an apparatus for analyte detection. The light source may be configured to emit a series of wavelengths for illuminating the sensor. In some embodiments, the plasmonic sensing chip containing the sensors may be inserted into the apparatus for analyte detection. The apparatus is configured to emit a light at a series of wavelengths onto the sensors, and to collect an optical spectrum of the light transmitted through. absorbed by. or reflected from the sensors. For example, the apparatus can perform absorbance/transmittance measurements. In some embodiments the measurements are made at wavelengths ranging from 500-1000 nm.

The method further comprises collecting data from the sensor. In some embodiments, the method comprises collecting absorbance data from the sensor. In some embodiments, the method comprises collecting transmittance data from the sensor. In some embodiments, the method comprises collecting extinction data from the sensor. In some embodiments, the method comprises collecting absorbance. transmittance, and/or extinction data of the sensor. In some embodiments, the method further comprises comparing collected data with a baseline data of the sensor prior to the sample exposure. In some embodiments, the method further comprises comparing at least one of the collected absorbance, transmittance, and/or extinction data with a baseline data of the sensor prior to the sample exposure. For example, the absorbance/transmittance measurements of functionalized sensors are made prior to exposure to the sample. The peak absorbance wavelength of the functionalized sensor (prior to bonding with a target analyte) is identified. The absorbance/transmittance of the sensors are made again after exposing to the sample, and a shift in peak absorbance can be observed if a target analyte is present in the sample and binds with the probe on the functionalized sensors. The shift represents the detection signal.

In some embodiments, an array of sensors in the plasmon-resonance sensing device 100 of any of the present embodiments is exposed to the sample. In some embodiments, at least a first sensor 101a in the array of sensors 101 comprises nanostructures conjugated with a first biological probe. In some embodiments, at least a second sensor 101b in the array of sensors 101 comprises nanostructures conjugated with a second biological probe. In some embodiments, at least a third sensor in the array of sensors comprises nanostructures conjugated with a third biological probe. In some embodiments, at least a fourth sensor in the array of sensors comprises nanostructures conjugated with a fourth biological probe. In some embodiments, at least a fifth sensor in the array of sensors comprises nanostructures conjugated with a fifth biological probe. In some embodiments, a “n” number of sensors in an array of sensors comprises nanostructures conjugated to an “n” number of biological probes, wherein “n” is any number from 1 to 2000. The biological probes conjugated to different sensors may be the same or different. In some embodiments. each sensor in the array can be conjugated to different biological probes for a multiplex sensing capability. In this configuration, multiple analytes can be detected simultaneously.

In some embodiments, at least a first sensor 101a in the array of sensors comprises nanostructures conjugated with a first biological probe and at least a second sensor 101b in the array of sensors comprises nanostructures conjugated with a second biological probe. In some embodiments. a first set of sensors in the sensor array is functionalized with a first biological probe, and a second set of sensors in the sensor array is functionalized with a second biological probe. In some embodiments, the first biological probe and the second biological probe are different. In some embodiments, the first biological probe and the second biological probe are the same. In some embodiments, the first biological probe and the second biological probe independently comprise one or more of a protein. peptide strand, amino acid. RNA strand, DNA strand, or and/or nucleotide. In some embodiments, the first biological probe and the second biological probe independently comprise one or more of a modified protein, modified peptide, modified amino acid, modified RNA strand, modified DNA strand, and/or modified nucleotide. In some embodiments, the first biological probe and the second biological probe independently comprise at least one of: a peptide-nucleic acid, an aptamer, an antibody, an antibody fragment, a complimentary DNA, and/or an enzyme. In some embodiments, first biological probe and the second biological probe are independently selected from the group consisting of a peptide-nucleic acid, an aptamer, an antibody, an antibody fragment, a complimentary DNA, and an enzyme.

The detection of analyte(s) is based on an optical phenomenon that occurs between a metal nanostructure and a dielectric—localized surface plasmon resonance (LSPR). LSPR is observed when the wavelength of incident light is larger than the size of the conductive nanostructures. The nanostructures result in highly confined electric fields of LSPR modes, which serve as a sensitive transducer to changes in the local dielectric environment (binding event). The nanostructures can be conjugated to/covalently functionalized with probes that can bind with target analytes. Upon binding with the target analyte(s). red shifts in the spectral peak can be observed. In some embodiments the amount of red shift may be observed as a function of target analyte concentration. In some embodiments, the sensors detect transmittance, reflectance, and/or absorbance at certain wavelength range.

In some embodiments, the sensors that have been exposed to the sample, thus having analyte(s) bound to selective biological probe on the sensors, can be further exposed to functionalized particles configured to bind to the sensors that have analyte(s) present and bound to the biological probe. The functionalize particles may be nanoparticles or microparticles. In some embodiments, the particles may be metal, polymer, glass, or any material with a high refractive index, for example, a refractive index of about 1.5 and higher. When the functionalized particles are bound to the sensors, it has the potential to improve both sensitivity and specificity of the sensors. Without being bound to the theory, the sensitivity improvements may be due to the fact that the functionalized particle increases the change in refractive index at the sensor surface in the presence of the analyte. The additional binding of the functionalized particles to the sensors may improve the sensor signal through a greater peak-shift in the optical measurement. Specificity improvements may be due to the fact that two selective binding events are required (i.e., first analyte must bind to the sensor, then the functionalized particle must bind to the sensor-bound analyte). In some embodiments, the functionalized particles are functionalized to bind to the analytes that have bound to the biological probes.

In some embodiments, a spectrum of the sensor comprising an array of functionalized nanostructures may be obtained prior to exposure to a sample. This may provide baseline data for the determination and analysis of an analyte binding event.

### Nanostructures Fabrication

Also disclosed herein is a method of making an array of nanostructures. The method comprises coating a photoresist layer onto a substrate, patterning the photoresist, and depositing a metallic layer over the patterned photoresist layer. In some embodiments, the substrate may be non-conductive, and a modified method may provide an improved result. The method comprises coating a conductive photoresist layer onto a non-conductive substrate, patterning the conductive photoresist layer via photolithography, depositing an adhesion layer over the patterned conductive photoresist layer, and depositing a metallic layer onto the adhesion layer. In some embodiments, patterning the conductive photoresist layer comprises exposing the photoresist layer to the electron beam to create a desired pattern. In some embodiments, the pattern should match the dimensions of and the spacing between the nanostructures. In some embodiments, the method may involve lithographic techniques, such as electron-beam lithography, UV photolithography, or nanoimprint lithography. In some embodiments, roll-to-roll manufacturing may be employed for making the sensor array.

For example, photolithography may be utilized to remove the portions of the photoresist layer where the nanostructures should be disposed/formed on the substrate, leaving the portion of the substrate where there should not be any nanostructure masked by the patterned photoresist layer. The patterned photoresist layer therefore has removed portions resembling the size, shape, and location of where the metallic nanostructures should be disposed. The portion of substrate is exposed at where the nanostructures will be formed. When the metallic layer is subsequently disposed over the patterned photoresist layer, some metallic layer would be disposed on the exposed portions of the substrate, and some the metallic layer would be disposed on the remaining photoresist that is masking the substrate.

The method further comprises lifting off the patterned photoresist layer. Lifting off the patterned photoresist layer also takes off the portions of the adhesive layer and the metallic layer disposed on the remaining patterned photoresist layer, leaving behind the portions of the adhesive layer that are in contact with the substrate and the portions of the metallic layer on that portions of the adhesive layer. In some embodiments, the adhesion layer comprises chromium. In some embodiments, the adhesion layer has a thickness of about 2 nm, about 3 nm, about 4 nm, about 5 nm, about 6 nm, about 8 nm, about 9 nm, or any thickness that is between about 2 and about 9 nm. In some embodiments, the adhesion layer has a thickness of about 5 nm. In some embodiments, the metallic layer comprises a single metal. In some embodiments, the metallic layer comprises a mixture of metals. In some embodiments, the metallic layer comprises gold, silver, aluminum, platinum or copper. In some embodiments. the metallic layer comprises gold. The thickness of the metallic layer would be the same as the thickness of the nanostructures on the substrate as disclosed herein.

The method disclosed herein provides an array of sensors comprising an array of nanostructures that are regularly spaced apart. The shape, dimensions, and the spacing of the nanostructures made by such method are the same as disclosed herein.

### Functionalization of Nanoplasmonic Sensing Chip

Disclosed herein is a method of making a functionalized nanoplasmonic sensing chip. The method comprises providing a substrate comprising an array of sensors, affixing a micro-well adaptor on top of the substrate so an array of micro-wells is over the array of sensors and aligned with each sensor, and forming one or more functionalized sensors in the array of sensors. Forming the one or more functionalized sensors includes delivering a first batch of reaction solutions into one or more micro-wells atop one or more sensors using an automatic pipetting system. and then subsequently removing the first batch of reaction solution from the one or more micro-wells using the automatic pipetting system. The automatic pipetting system includes an array of pipets that can be loaded with one or more reaction solutions. In some embodiments, the array of pipets may be loaded with two or more different reaction solutions, thus allowing delivery of two or more different reaction solutions to the array of micro-wells/sensors. The array of pipets may also be used to remove the reaction solutions from some or all of the micro-wells/sensors after the reactions. The array of pipets can deliver or remove reaction solutions from a specific micro-well/sensor or a specific group of micro-wells/sensors. In some embodiments, each reaction solution may include one or more reagents for modifying the array of nanostructures in the sensor. In some embodiments, each reaction solution may include one or more biological probes.

In some embodiments, multi-step reactions may be utilized for functionalizing the sensors. Thus, forming one or more functionalized sensors may further involve delivering a second batch of reaction solutions into the one or more micro-wells, and subsequently removing the second batch of reaction solutions from the one or more micro-wells, wherein the delivering and removing the second batch of reaction solutions are performed by an automatic pipetting system.

In some embodiments, the first batch of reaction solutions comprises two or more different reaction solutions. In some embodiments, the second batch of reaction solutions may also comprise two or more different reaction solutions. In some embodiments, the reaction solutions may include different biological probes. Thus the array of functionalized sensors may comprise two or more different biological probes. For example, some of the functionalized sensors in the array may comprise a specific biological probe, while other functionalized sensors comprise a different biological probe. In some embodiments, each of the functionalized sensor may comprise different biological probes. In some embodiments, a reaction solution may include one or more biological sensors. Thus each functionalized sensor may comprise one or more biological probes. One or more biological probes can conjugate to the array of nanostructures in each sensor. In some embodiments, the sensor may comprise one, two, three, four, or more biological probes configured to bind to one or more analytes.

Then the method further includes removing the micro-well adaptor from the substrate. In some embodiments, the one or more sensors arc functionalized with a biological probe while the first batch of reaction solutions in the one or more micro-wells is in contact with the sensors. In some embodiments, the one or more sensors is functionalized with a biological probe after two or more reaction steps. In some embodiments, the sensor (e.g., the one or more sensors) each comprises an array of nanostructures disclosed herein.

In some embodiments, the automatic pipetting system can be configured to deliver different reaction solutions to multiple micro-wells for functionalizing multiple sensors in the array. In some embodiments, multiple reaction solutions are delivered to different sensors in the array, thereby functionalizing multiple sensors substantially at the same time. In some embodiments, the automatic pipetting system can be configured to removing different reaction solutions from multiple micro-wells. In some embodiments, multiple reaction solutions are removed from different sensors in the array substantially at the same time. In other embodiments, some reaction solutions may be removed at a different time to allow longer or shorter reaction time.

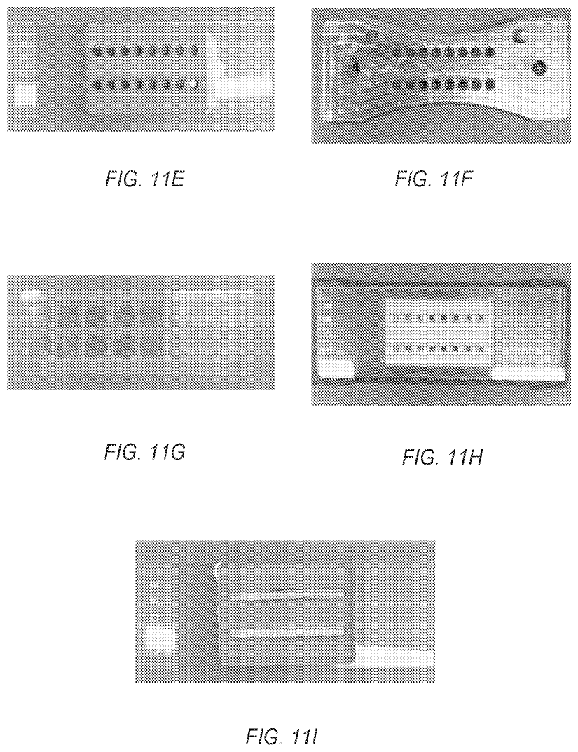

FIGS. 11A-11B depict two alternative views of a 3D printed mold for a fabricated polymer well shown in FIGS. 11C-11D. Other embodiments of the micro-wells are shown in FIGS. 11E-11I.

In some embodiments, additional pre-treatment step(s) can be performed prior to delivering any reaction solution. The pre-treatment step may include washing the nanostructure surface, wetting the nanostructure surface, or activation the nanostructure for subsequent reaction/functionalization. In some embodiments, the method may further comprise delivering an activation solution into at least a portion of the micro-wells atop the sensors in the array using an automatic pipetting system; and subsequently removing the activation solution prior to delivering a reaction solution.

The method disclosed herein provides at least one functionalized sensor comprises an at least one biological probe. In some alternatives, the first functionalized sensor comprises a first array of nanostructures conjugated to a first biological probe. In some alternatives, the second functionalized sensor comprises a second array of nanostructures conjugated to a second biological probe. In some alternatives, additional sensors comprising a nanostructures array may be conjugated to additional biological probe(s), up to the number of sensors in the sensor array. For example, a “n” number of sensors in an array of sensors comprises nanostructures conjugated to an “n” number of biological probes, wherein “n” is any number from 1 to 2000. In some embodiments, n may be any number from 1 to 1000, from 1 to 500, from 1 to 100, or from 1 to 25.

Each of the biological probes is independently selected from the group consisting of a peptide-nucleic acid (PNA), an oligonucleotide, an aptamer. an antibody, an antibody fragment, a complimentary DNA, and an enzyme. In some alternatives, the first biological probe and the second biological probe are independently selected from the group consisting of a peptide-nucleic acid, an aptamer, an antibody, an antibody fragment, a complimentary DNA, and an enzyme. In some alternatives, the first biological probe and the second biological probe are different. In some alternatives, the first biological probe and the second biological probe are the same. In some embodiments, each sensor may be functionalized with a different biological probe. In some embodiments, some of the sensors in the array may be functionalized with different biological probes. In some embodiments, all the sensors in the array may be functionalized with the same biological probe.

In some embodiments, reaction solutions are delivered to all the micro-wells simultaneously. In some alternatives, reaction solutions are subsequently removed from the micro-wells simultaneously. In some alternatives, reaction solutions are removed from the micro-wells at a different time to accommodate for different reaction time for functionalizing the sensors with a variety of the biological probes. In some embodiments, reaction solutions can also be delivered to different micro-wells at a different time. In some alternatives, the first reaction solution and the second reaction solution are delivered to the first micro-well and the second micro-well simultaneously, and subsequently the first reaction solution and the second reaction solution are removed from the first micro-well and the second micro-well. In some embodiments, delivering and removing a reaction solution may be performed by an automatic pipetting system. In some embodiments, the automatic pipetting system may be configured to remove different reaction solutions at a different time. In some embodiments, the automatic pipetting system may be configured to deliver different reaction solution at a different time.

In some embodiments, the nanostructures comprise a metal. In some alternatives, the nanostructures comprise a single metal. In some alternatives, the nanostructures comprise a mixture of metals. In some alternatives, the nanostructures comprise gold, platinum. aluminum, silver, or copper. In some alternatives, the nanostructures comprise gold.

### Functionalized Plasmonic Sensing Chip

Functionalized plasmonic sensing chips comprising an array of functionalized sensors are disclosed. In some embodiments, each of the functionalized sensors in the array comprises an array of nanostructures conjugated to at least one biological probe. In some embodiments, the array of nanostructures is conjugated to two or more biological probes configured to bind to two or more analytes. The biological probe is configured to bind to at least one analyte. In some alternatives, the at least one biological probe independently comprises at least one of: a peptide-nucleic acid, an oligonucleotide, an aptamer, an antibody, an antibody fragment, a complimentary DNA, and/or an enzyme. In some embodiments, the biological probe is independently selected from the group consisting of a peptide-nucleic acid, an oligonucleotide, an aptamer, an antibody, an antibody fragment, a complimentary DNA, and an enzyme. In some embodiments, all functionalized sensors in the array comprise the same biological probes. In some alternatives, at least one of the functionalized sensors in the array comprises at least one different biological probe from the others. For example, some of the functionalized sensors in the array may comprise a specific biological probe, while other functionalized sensors comprise a different biological probe. In some embodiments, each of the functionalized sensors in the array comprise at least one different biological probe. One or more biological probes can conjugate to the array of nanostructures in each sensor. In some embodiments, the functionalized sensor may comprise one, two, three, four, or more biological probes configured to bind to one or more analytes.

In some embodiments, the functionalized plasmonic sensor chip may include 1 to 100 (and any numbers in between) different biological probes. For example. the functionalized plasmonic sensor chip may include 1, 2, 3, 4, 5, 6, 7, 8. 9, 10, 11, 12, 13. 14, 15, 20, 24, 30, 36, 40. 48, 50, 54, 60, 70, 80, 90, or 100 different biological probes. In some embodiments, each functionalized sensor in the functionalized plasmonic sensor chip may contain different biological probe(s). In some embodiments, the array of the nanostructures in each sensor may conjugate to one or more biological probes, and the one or more biological probes may be different.

In some embodiments, the nanostructures comprise a metal. In some alternatives, the nanostructures comprise a single metal. In some alternatives, the nanostructures comprise a mixture of metals. In some alternatives, the nanostructures may comprise gold, platinum, aluminum, silver, or copper. In some alternatives, the nanostructures comprise gold. In some alternatives, the nanostructures in the array are regularly spaced apart and may have the geometry described herein.

### Multiplex Analyte Detections

A method for detecting two or more analytes simultaneously is also described. In some alternatives, the method may detect 3, 4, 5, 6, 7, 8, 9. 10. 11. 12, 13, 14, 15, 20, 24, 30, 36, 40, 48, 50, 54, 60, 70, 80, 90, and/or 100 analytes. In some embodiments, up to 50 analytes are detected. In some embodiments, up to 24, up to 50, up to 80, or up to 100 analytes may be detected. The method comprises exposing the array of functionalized sensors on the plasmonic sensing chip of any of the alternatives disclosed herein to a sample. The functionalized sensors are configured to detect the presence of certain target analytes. In some embodiments, the functionalized sensor may be configured to identify or detect various markers, subtypes, strains, genotypes and/or variants of a biological species. When the functionalized sensors are exposed to the sample, one or more target analytes, if present, bind to the corresponding biological probes. The binding event causes a change in the local dielectric environment of the sensors. The sample may comprise a bodily fluid. such as blood, urine, or saliva, etc. In some embodiments, the sample may be evacuated or removed from the functionalized sensors following the exposure step.

Optionally, the array of functionalized sensors may be subject to a heating step after the exposure to the sample. In some embodiments, the array of functionalized sensors is heated up to about 85° C. or any temperature between 25° C. and 85° C. In some embodiments, the array of functionalized sensors may be exposed to heat before. during, or after subsequent steps. In some embodiments, the array of functionalized sensors may be exposed to heat before, during, or after the measurement.

The method further comprises illuminating a light at a series of wavelengths onto the functionalized sensors; and collecting absorbance, transmittance, and/or extinction data from the functionalized sensors. The light may be emitted from a light source in an apparatus for analyte detection. The light source may be configured to emit a series of wavelengths for illuminating the sensors. In some embodiments, the plasmonic sensing chip containing the functionalized sensors may be inserted into the apparatus for analyte detection. The apparatus is configured to emit a light at a series of wavelengths onto the functionalized sensors, and to collect an optical spectrum of the light transmitted through, absorbed by, or reflected from the sensors.

In some embodiments, the method further comprises comparing collected data with a baseline data of the sensors prior to the sample exposure. The baseline data for a functionalized sensor can be collected using the apparatus for analyte detection described above. In some embodiments, the baseline data can be collected prior to exposure of the sensor to the sample. In some embodiment, the baseline data is provided for a sensor functionalized with a specific biological probe. A shift in the spectral peaks after the sample exposure indicates the binding of the target analyte with the biological probe, therefore indicating the presence the target analyte in the sample. In some embodiments. the amount of the spectral peak shift may further be interpreted to provide a quantitative or semi-quantitative measurement of the concentration of a target analyte in the sample.

In some embodiments where the sensors in the array are functionalized with different biological probes, exposure of the array to the sample can result in binding of various target analytes to the corresponding sensors. Illuminating the array of sensors with a light at a series of wavelengths would allow the collection of optical spectra of each sensor be collected and compared with the baseline data. One exposure of the sensing device chip could allow detection and identification of different target analytes.

In some embodiments, the plasmon-resonance sensing device enables point-of-care (POC) detection of target analytes and POC diagnosis of disease(s)/condition(s). In some embodiments, rapid results (about 15 min or less) may be provided.

### Methods for Detecting HPV

HPV may be detected using a sensor comprising a biological probe designed to target a nucleic acid sequence derived from a HPV pathogen. The method includes exposing the sensor to a sample that may contain a nucleic acid sequence derived from one or more HPV pathogens, and collecting electrical, fluorescent, absorbance, transmittance, and/or extinction data from the sensor. In some embodiments, the biological probe may be a peptide nucleic acid (PNA) probe or an oligonucleotide probe.

In some embodiments, the sensor may comprise one or more biological probes. In some embodiments, each of the biological probes may be designed to bind different target nucleic acid sequences. As such, the sensor may be able to detect multiple or various target nucleic acid sequences at once. For example, the sensor can detect the presence of any of the different HPV pathogens and confirm the patient's HPV diagnosis. That means HPV may be diagnosed regardless of which of the various HPV pathogens is present. In some embodiments, the sensor may be able to detect and identify one or more specific HPV pathogens in a patient. This information may be useful for determining a proper or the most effective treatment option.

In some embodiments, a single biological probe can bind nucleic acids derived from more than one HPV pathogens. In some embodiments, a single biological probe can bind more than one nucleic acid derived from one high-risk HPV genotypes. In some embodiments, the biological probe may be designed to bind nucleic acid sequences from more than one HPV varieties or mutations. As a result, a sensor comprising one or more biological probes may be able to detect multiple HPV species, strains, mutants, segments, particles or derivatives. In some embodiments, a sensor comprising one or more biological probes may be able to identify one or more HPV pathogens and derivatives.

The biological probe may be designed or selected using computational and/or bioinformatic methods. These methods allow for rational selection of probe sequences that align upon known sequences in the scientific literature. In some embodiments, the computational approaches utilized custom python scripts, open-access sequence databases, and thermodynamic modeling tools. In some embodiments, the biological probes contain intentionally varying degrees of mismatch with the target nucleic acids. These mismatches allow for an additional degree of freedom when measuring the presence of a target nucleic acid.

In some embodiments, the biological probes described herein independently has a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

The sensor may have a physical property that changes upon the binding of one or more target nucleic acid sequences to the biological probes associated with the sensor. The change in the physical property can be detected by the change in electrical, fluorescent, absorbance, transmittance, and/or extinction measurement. Some non-limiting examples of sensors may include electrochemical sensors, fluorescence-based sensors, resistive sensors, and optical sensors.

### Nanoplasmonic Sensor for Pathogen/HPV Detection

A nanoplasmonic sensor is an example of the sensor that can be functionalized with a biological probe for detecting human papillomavirus pathogens. In some embodiments, the nanoplasmonic sensor comprises an array of functionalized sensors, wherein each of the functionalized sensors in the array comprises an array of nanostructures conjugated to a biological probe/capture ligand, such as a peptide nucleic acid (PNA) probe or an oligonucleotide probe. In some embodiments, the biological probe is configured to detect the presence of a pathogen associated with human papillomavirus (HPV). In some embodiments, the biological probe is configured to detect the presence of human papillomavirus. In some embodiments, the Biological probe is configured to detect the presence of a human papillomavirus pathogen using a specific marker associated with that given pathogen. In some embodiments, the specific marker is derived from the human papillomavirus. In some embodiments, the specific marker is from a subject's response to infection by the human papillomavirus. In some embodiments, the nanoplasmonic sensor is configured to simultaneously detect multiple strains, segments, particles, mutants, and/or species of the human papillomaviruses.

In some embodiments, more than one functionalized sensors in the array are capable of detecting a human papillomavirus in a sample. In some embodiments, at least two of the functionalized sensors in the array comprise the same biological probe for detecting a human papillomavirus. In some embodiments, at least two of the functionalized sensors in the array comprise the same biological probe for detecting the same marker for an human papillomavirus. In some embodiments, all of the functionalized sensors in the array comprise the same biological probe for detecting a human papillomavirus. In some embodiments, all of the functionalized sensors in the array comprise the same biological probe for detecting the same marker for human papillomavirus.

In some embodiments, at least one of the functionalized sensors in the array comprises a different biological probe for detecting a different strains, segments, particles, mutants, and/or species of the human papillomaviruses from the other functionalized sensors. In some embodiments, at least one of the functionalized sensors in the array comprises a different biological probe for detecting a different marker for a human papillomavirus from the other functionalized sensors. In some embodiments, different markers may be used to detect the same human papillomavirus. In some embodiments, different markers may be used to detect a different human papillomavirus. In some embodiments, different markers may be used to detect different strains, segments, particles, mutants, and/or species of human papillomavirus. In some embodiments, all of the functionalized sensors in the array comprise different biological probes from one another for detecting different strains, segments, particles, mutants, and/or species of human papillomavirus. In some embodiments, the nanoplasmonic sensor is configured to simultaneously detect multiple strains, segments, particles, mutant, and/or species of human papillomavirus. In some embodiments, each of the functionalized sensors in the array comprises a different biological probe.

In some embodiments, a functionalized sensor may be functionalized with a negative control biological probe. The negative control biological probe may be designed to be complementary to a synthetic sequence of DNA/RNA that does not exist naturally. The negative control functionalize sensor will be expected to always return a negative result.

In some embodiments, a functionalized sensor may be functionalized with a positive control biological probe. The positive biological probe would be complementary to a synthetic sequence of DNA. A low concentration of that sequence of DNA may be spiked into the sample early in the reaction. This will indicate if the sample prep and fluid handling do enable a known concentration of target DNA to reach the sensor, indicating successful assay operation.

It will be understood that a human papillomavirus can be any virus or viral component associated with the human papillomavirus viral family. Non-limiting examples of HPV include HPV18, HPV16, hrHPV, HPV type 16, HPV type 18, HPV type 31, HPV type 33, HPV type 35, HPV type 39, HPV type 45, HPV type 51, HPV type 52, HPV type 56, HPV type 58, HPV type 59, HPV type 66, HPV type 68, and any derivative/mutant strain thereof.

It will be understood that the biological probe can comprise any peptide and/or nucleic acid sequence capable of binding to/associating with a segment of a human papillomavirus. In some embodiments, the sequence is complementary to a sequence present on/in the human papillomavirus. In some embodiments, the sequence directly recognizes the human papillomavirus. In some embodiments, the biological probe comprises one or more of a protein, peptide strand, amino acid, RNA strand, DNA strand, or and/or nucleotide. In some embodiments, the biological probe comprises one or more of a modified protein, modified peptide, modified amino acid, modified RNA strand, modified DNA strand, and/or modified nucleotide. In some embodiments, the probe comprises at least one of: a peptide-nucleic acid, an aptamer, an antibody, an antibody fragment, a complementary DNA, and/or an enzyme. In some embodiments, the probe is selected from the group consisting of a peptide-nucleic acid, an aptamer, an antibody, an antibody fragment, a complimentary DNA, and an enzyme. In some embodiments, the biological probe may be a PNA probe or an oligonucleotide probe having a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8. The probe sequences are listed in Table 5.

The nanostructure arrays are as disclosed herein. The nanostructures comprise a metal. In some embodiments, the nanostructures comprise a single metal. In some embodiments, the nanostructures comprise a mixture of metals. In some embodiments, the nanostructures comprise silver. In some embodiments, the nanostructures comprise copper. In some embodiments, the nanostructures comprise gold. The nanostructures in the sensors can be functionalized with the biological probes using the automatic pipetting system and method as described herein.

Also disclosed herein is a method for detecting the presence of one or more human papillomaviruses. In some embodiments, the method comprises exposing the nanoplasmonic sensor of any of the present embodiments disclosed herein to a sample, illuminate a light at a series of wavelengths onto each of the functionalized sensors, and collecting absorbance. transmittance or extinction data of each of the functionalized sensors. In some embodiments, the sample is a bodily fluid sample of a patient suspecting of having a human papillomavirus infection. In some embodiments, the bodily fluid sample is mucus, cells, and cell debris, which may be collected using cervical swabs or vaginal swabs. In some embodiments. the light for eliminating the functionalized sensors may be emitted from a light source in an apparatus for analyte detection. The light source may be configured to emit a series of wavelengths for illuminating the sensor. For example, the series of wavelengths includes wavelengths ranging from 500-1000 nm.

In some embodiments, the method further comprises comparing the collected absorbance, transmittance, and/or extinction data of each functionalized sensor with a baseline data of each of the functionalized sensors prior to exposure to the sample. In some embodiments, the comparing step reveals an optical peak shift when an at least one human papillomavirus is detected. The baseline data of the functionalized sensor includes the absorbance/transmittance measurements of functionalized sensors made prior to exposure to the sample. The peak absorbance wavelength of the functionalized sensor (prior to bonding with a target analyte) is identified. The absorbance/transmittance of the sensors are made again after exposing to the sample, and a shift in peak absorbance can be observed if a target analyte. such as human papillomavirus or particle of a human papillomavirus, is present in the sample and binds with the probe on the functionalized sensors. The shift represents the detection signal. In some embodiments, the amount of the optical peak shift is correlated to the concentration of pathogen in the sample. In some embodiments, the amount of the optical peak shift is correlated to the concentration of the human papillomavirus or particle of a human papillomavirus in the bodily fluid sample.

In some embodiments, two or more of the functionalized sensors may comprise the same biological probe. In some embodiments, at least one of the functionalized sensors may comprise different biological probes. In some embodiments, each of the functionalized sensors may comprise different biological probes. In some embodiments, when one or more of the functionalized sensors in the array comprises different biological probes. multiple strains or species of the human papillomavirus or particle of a human papillomavirus can be detected simultaneously (i.e., with the same nanoplasmonic sensor/test kit). In some embodiments, the method may be performed at the point of care—that is at the location of the patient care, such as at the physician's offices, clinics, hospitals, long-term-care facilities, or patient's home, etc.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example. reference to “a sequence” may include a plurality of such sequences, and so forth.

The terms “comprising,” “including,” “containing,” and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

The term “nanostructure,” as used herein, has its standard scientific meaning and thus refers to any structure that is between about molecular size, to about microscopic size. Nanostructures comprise nanomaterials, which can be any material in which a single unit is sized at about 1 nm to about 200 nm. Nanostructures include nanoparticles, nanorods, nanosquares, nanocubes, gradient multilayer nanofilm (GML nanofilm), icosahedral twins. nanocages, magnetic nanochains, nanocomposite, nanofabrics, nanofiber, nanoflower, nanofoam, nanohole, nanomesh, nanopillar, nanopin film, nanoplatelet, nanoribbon, nanoring, nanobipyramids, irregular nanoparticles, nanosheet, nanoshell, nanotip, nanowire, and nanostructured film. It will be understood that a nanostructure can have various geometric shapes and properties based on the components of that nanostructure.

The term “analyte” refers to a substance or chemical constituent that is of interest. For examples, analyte may include biological or chemical substance that may be detected by a sensing device and may be of interest for diagnosing a disease or a condition.

All patents and other publications; including literature references, issued patents, published patent applications. and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The technology described herein is further illustrated by the following examples which in no way should be construed as being further limiting.

### Example 1: Electromagnetic Simulation

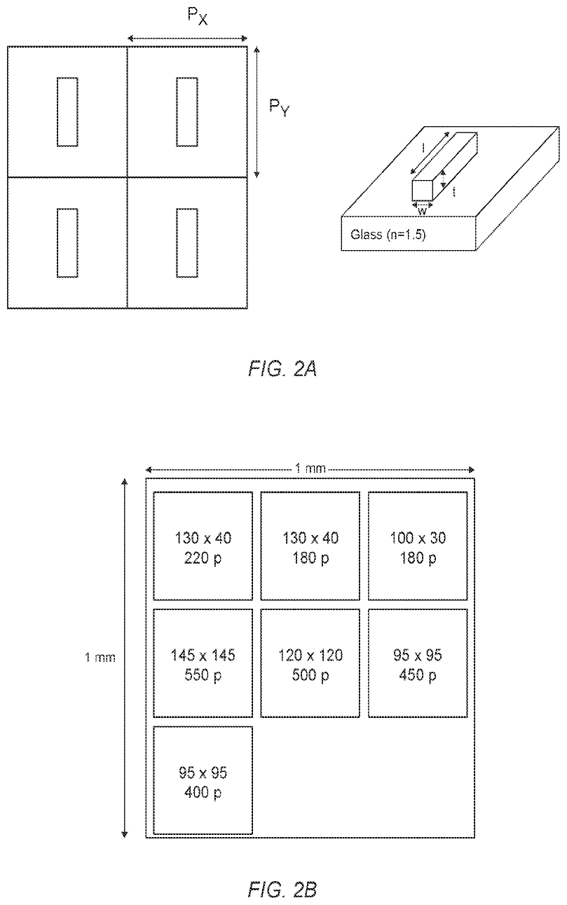

Several geometries for simulation and testing included some nanorods and some coupled nanoarrays. The nanorods is designed to reflect randomly oriented colloidal nanorods dispersed onto a glass slide. The coupled nanoarrays are designed to create surface lattice resonances. The seven geometries for a fabrication dose test are as shown in Table 1 and FIGS. 2A and 2B. FIG. 2A shows a grid with labeled dimensions for length (l), width (w), thickness (t), and spacing/periodicity (p) of the nanorods. FIG. 2B is a map of arrangement of the nanorod array within a sensor unit. As show in Table 1, the test geometries T1-T3 are nanorods and the test geometries T4-T10 are coupled nanoarrays.

TABLE 1
Test Geometries. Length, width, periodicity, and thickness of the dose matrix test.
All dimensions are listed in nanometers.

| Sample | Length (1), nm | Width (w), nm | Thickness (t), nm | Spacing (p), nm |
|---|---|---|---|---|
| T1 | 130 | 40 | 40 | 220 |
| T2 | 130 | 40 | 40 | 180 |
| T3 | 100 | 30 | 40 | 180 |
| T4 | 145 | 145 | 40 | 550 |
| T5 | 120 | 120 | 40 | 500 |
| T6 | 95 | 95 | 40 | 450 |
| T7 | 95 | 95 | 40 | 400 |
| T8 | 95 | 95 | 50 | 450 |
| T9 | 120 | 120 | 40 | 500 |
| T10 | 145 | 145 | 40 | 550 |

Full-wave electromagnetic simulations were conducted using Lumerical photonic simulation software. Periodic boundary conditions were applied in the x- and y-dimensions for each of the geometries T1-T7 as shown in FIGS. 2A-2B. For bulk sensing experiments, the refractive index of the surrounding media was changed. For PNA-DNA binding experiments, conformal shell layers of defined refractive index were modeled atop the nanostructures. Extinction and transmittance curves were returned for the wavelength range of 400-1200 nm.

### Example 2: Simulation Setup and Defining Figure-of-Merit

To study the plasmonic resonance shape as well as sensitivity to changes in refractive index, initial simulations included a bulk refractive index sensitivity analysis. In an initial iteration, gold nanorods with a wide spacing designed to represent the ordered nanoarrays was tested.

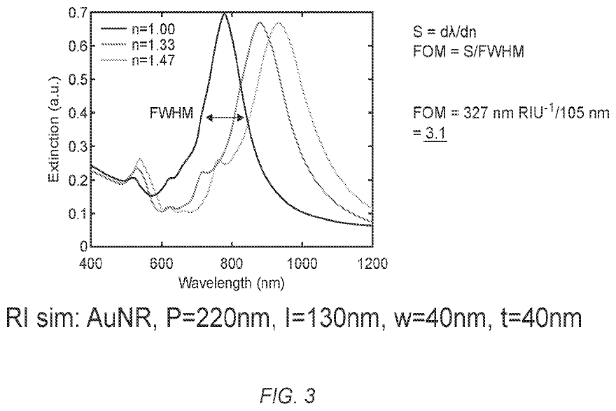

The resonances were modeled in air, water, and glycerol (increasing refractive index) and the peak locations were calculated for each of the extinction curves. This allowed for development of a sensor figure-of-merit (FOM) that considers the peak shift(s) and the narrowness of the resonances (full width at half maximum-FWHM) as shown in FIG. 3. The figure of merit was defined as the shift over full width at half maximum, allowing for a direct comparison between various geometries. A larger figure of merit represents better sensing performance due to (1) larger peak shifts for the same refractive index change, and (2) easier discrimination of peak shifts due to a narrow resonance curve. This analysis was repeated for all geometries considered.

### Example 3: PNA-DNA Binding Simulation

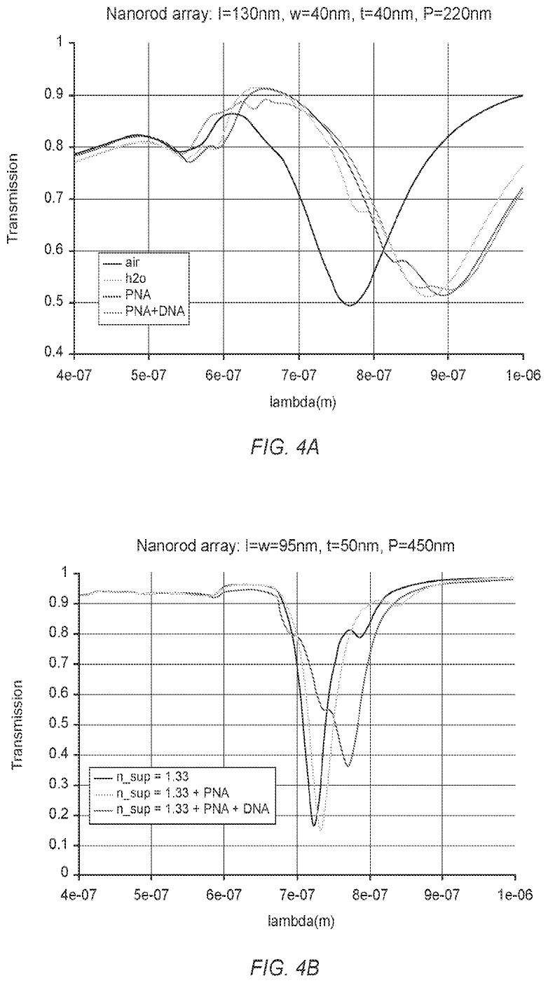

Another method of simulating these nanostructures involved simulating conformal layers with the same refractive indices expected of peptide nucleic acid (PNA) probes and PNA probes bound to DNA. We observed that the shift upon PNA+DNA binding for the surface lattice geometry (as shown in FIG. 4B) is much more apparent than the shift for the disperse nanorod geometry (as shown in FIG. 4A). These simulations point to expected shifts associated with DNA biosensing for each geometry.

### Example 4: Nanosensor Fabrication

Electron-beam lithography is a common method for patterning precise nanoscale features onto a substrate. Typically, such patterns are processed onto silicon wafers, which are optically opaque and highly conductive. For the transmittance-mode operation of the sensor, the nanostructures were configured to sit atop a transparent quartz wafer. A protocol for nanoscale patterning onto a transparent, non-conductive surface was developed.

First a thin layer of a conductive photoresist was spin-coated on the transparent quartz wafer before exposure to the pattern with an electron beam (JEOL E-beam microscope). After this, a thin (˜5 nm) chromium adhesion layer was thermally evaporated onto the patterned substrate. followed by a thicker (about 40-50 nm) pure gold layer. Chemical liftoff was conducted to form the nanostructures array before dicing the substrate for testing. The first sample produced with this pattern was a dose matrix test to evaluate the power of the electron beam. After this parameter was identified, all future processes were conducted under the same conditions.

### Example 5: Simulation of Selected Geometries

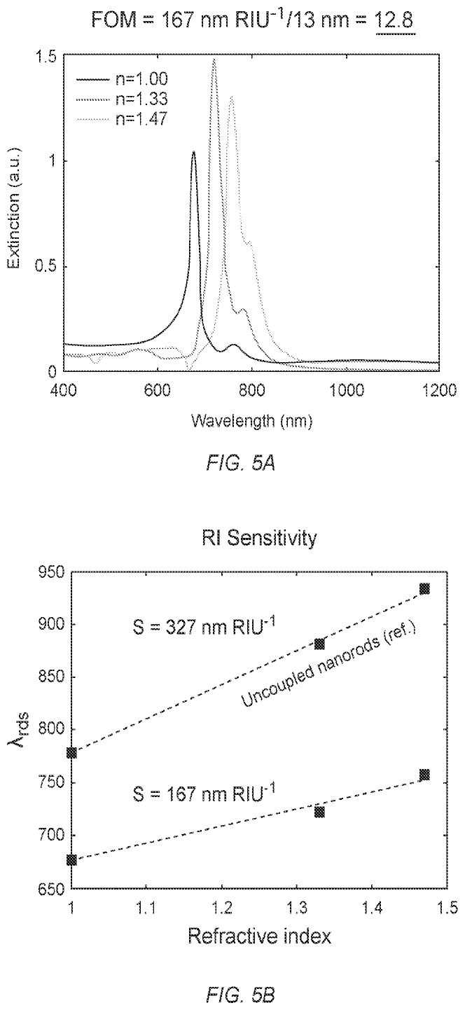

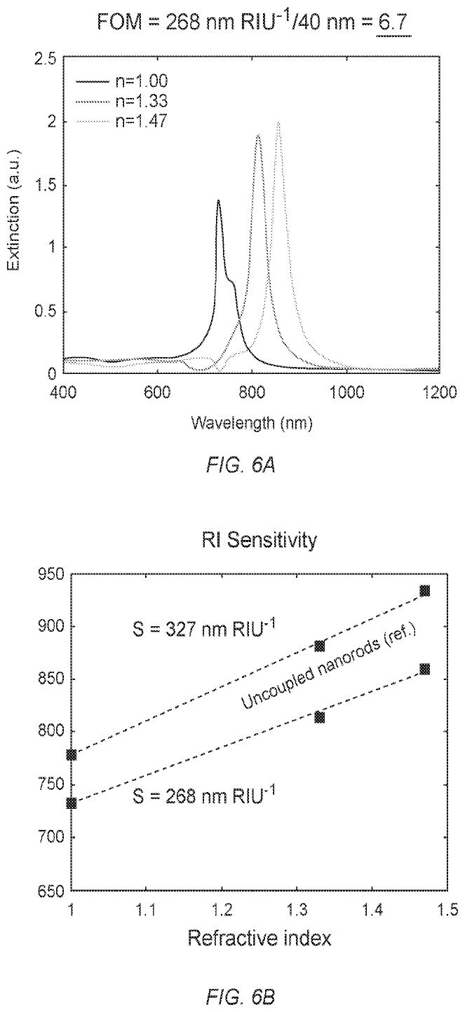

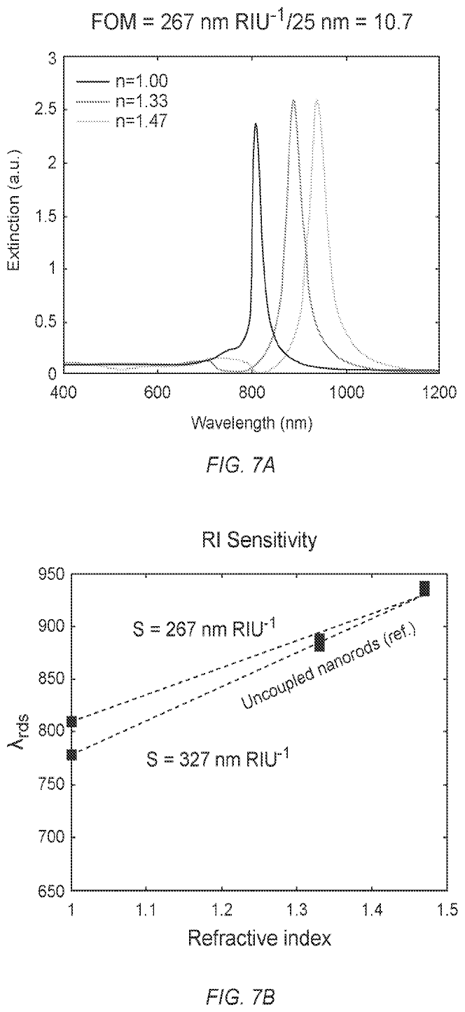

Bulk refractive simulations were conducted on sample geometries T8-T10 described in Table 1. Transmittance through the samples was measured using an optical readout instrumentation. Wavelength bounds were set from 450 nm to 950 nm. For seamless integration with the readout instrumentation, the individual sensor of the sensing device was fabricated to have a 1 mm² area of nanostructures array to fully align with the light source spot size and minimizing signal loss. The results for the three surface lattice resonance geometries (T8-T10) are as shown in FIGS. 5A/B, 6A/B, and 7A/B, respectively. Both the shape of the peak and the refractive index peak shifts are shown. The calculated figure-of-merits for T8-T10 were 12.8, 6.7, and 10.7, respectively. Further, the refractive index sensitivities of each of these geometries are shown in FIGS. 5B, 6B, and 7B. All sensitivities are compared to the 140 nm×40 nm 220p sample labeled “uncoupled nanorods”. A higher slope indicates better sensing performance. Sample geometry T10 is the highest performance due to its high figure of merit (10.7) and its relatively high refractive index sensitivity (267 nm/RIU).

### Example 6: Comparison of Simulation and Experiment

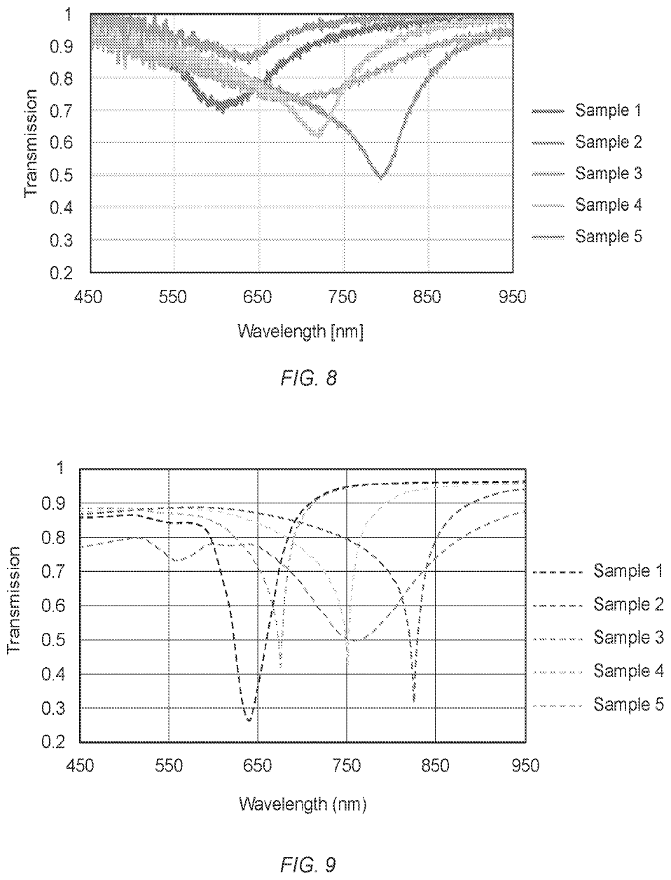

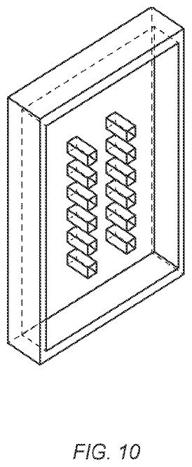

Nanostructure array samples 1-5 were fabricated with the nanostructure dimensions shown in Table 2. The transmittance of each sample was experimentally measured (shown in FIG. 8) and compared to the peak shape from the simulations (shown in FIG. 9). There was found to be exceptional agreement between the experimental and simulation data, including the peak shape and resonance location.

TABLE 3
Dimensions of nanostructure arrays.

| Sample | Length (1), nm | Width (w), nm | Thickness (t), nm | Spacing (p), nm |
|---|---|---|---|---|
| 1 | 96 | 96 | 50 | 400 |
| 2 | 145 | 145 | 50 | 550 |
| 3 | 130 | 130 | 50 | 220 |
| 4 | 120 | 120 | 50 | 500 |
| 5 | 95 | 95 | 50 | 450 |

The present disclosure also puts forth a methodology for rational design of regularly spaced nanoparticle arrays for plasmonic sensing. The Applicants tested 5-7geometries through both simulation and experimental analysis, and finally selected the 145 nm×145 nm Through both simulation and experimental analysis, a nanoarray geometry that shows high-amplitude resonance and refractive index sensitivity may be selected for the production of the plasmonic-resonance sensing device.

### Example 7: Functionalization of Nanostructures

A 2×6 array of 1 mm² sensors (12 sensors total) was functionalized with peptide-nucleic acid (PNA) probes. Each of the sensors contains an array of 145 nm×145 nm gold nanostructures with regular spacing. In order to individually functionalize the sensor arrays to be target-specific. a polydimethylsiloxane (PDMS) polymer micro-well array was fabricated. This micro-well array was aligned with the substrate such that each sensor could be accessed through a single micro-well. This approach created repeatable, programmable coordinates for the automatic pipetting system (e.g., Integra ASSIST PLUS pipetting robot).

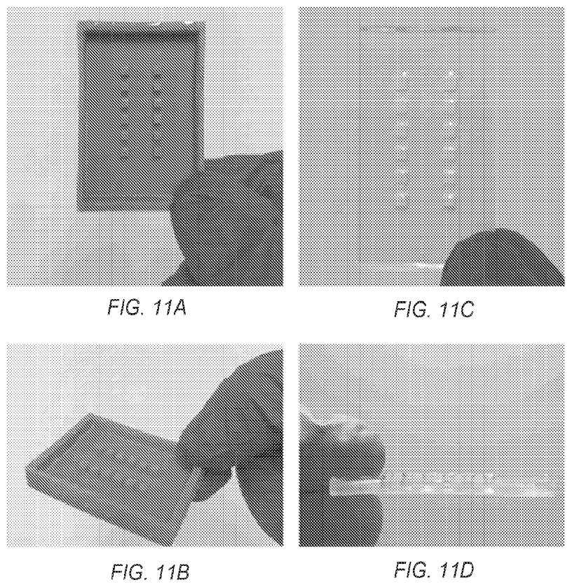

The micro-well structure atop the sensing array allowed for individual fluid delivery to each sensing spot, enabling multiplexing of up to 12 targets on a single sensing chip. To this end, a mold was designed using Solidwaorks CAD to allow for fabrication of a polymer micro-well array that align with the coordinates of the sensors (FIG. 10). The mold for casting the PDMS micro-wells was designed in Solidworks consisting of twelve 2 mm×2 mm×5 mm (20 mm³) pillars. The pillars were positioned to match the coordinates of sensor array on the glass substrate. Master molds, as shown in FIGS. 11A and 11B. were then made using SLA 3D printing.

Micro-well array devices were fabricated in the molds using PDMS soft lithography. Sylgard 184 silicone elastomer, base and curing agent (Dow Corning, Midland, MI) were mixed in a ratio of 10:1, by weight. Next, the PDMS prepolymer was cast on the master mold and cured at 80° C. in a convection oven for approximately 1.5 h. The cured PDMS micro-well array, as shown in FIGS. 11C and 11D, was removed from the master mold. The polymer micro-well array was affixed atop the sensor array using washable glue, enabling removable bonding for sensor reuse. This entire system was attached to a standard 75×25 mm microfluidic chip and was then ready for molecular detection.

### Example 8: Automated Robotic Functionalization of Sensors

The prepared plasmonic sensing chip was integrated with the automatic pipetting system (e.g., Integra ASSIST Plus) for surface functionalization. To covalently functionalize the sensor with selected biological probes, such as PNA probes, the gold nanostructures on a glass substrate were first incubated with 1 mg/mL dithiobis succinimidyl propionate (DSP) dissolved in dimethyl sulfoxide (DMSO) for 20 min. This crosslinking molecule activated the gold surface to enable coupling of free amines on the PNA. Next, the sensor arrays were put in contact with 1 mg/mL PNA probe dispersed in Tris-EDTA buffer (pH 7.0) for 30-45 min. Transmission spectra were collected before and after conjugation to characterize successful PNA conjugation.

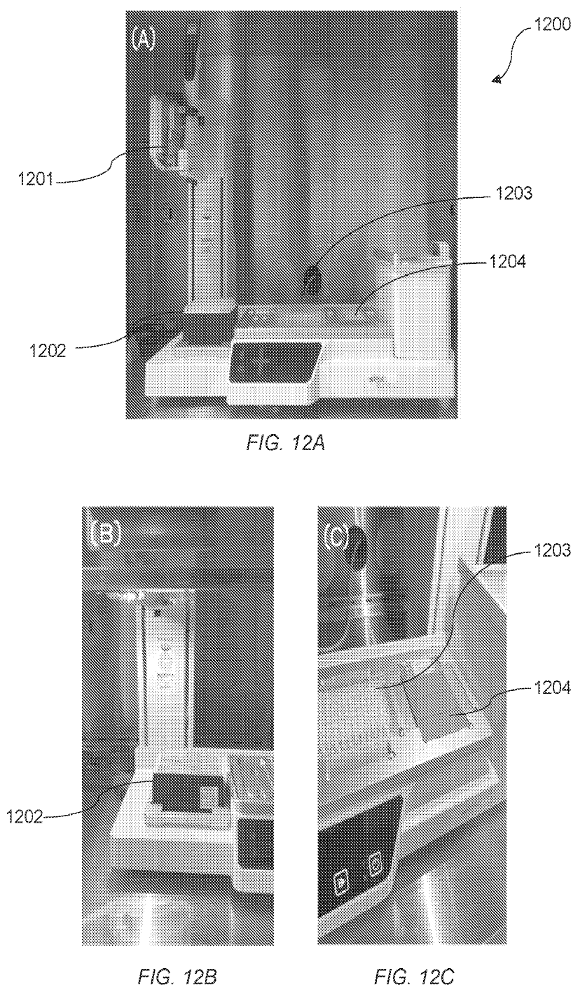

The sensor functionalization process described above was automated using an Integra ASSIST PLUS pipetting robot. In order to effectively position the device onto the deck of the Integra ASSIST PLUS pipetting robot, we designed and fabricated (3D printed) a custom 4-slot microscope slide holder/adaptor the size of a standard 96-well plate. This adapter could readily be integrated with the liquid handler's robotic deck. A 96-well plate was pre-loaded with functionalization reagents and placed in the robot's aspiration deck. To start up the machine, a Voyager electronic 125 μL, 8-channel pipette was loaded onto the robot. A suite of six programs were developed to aspirate, dispense, and clear tips in an automated fashion. These custom programs allow for multiplexed functionalization of twelve PNAs upon the sensor arrays. Table 4 shows 6 programs for automated functionalization of the sensors using the pipetting robot. The programs indicate pipette tip location, 96-well plate location, aspiration volume, and dispense volume for each stage. FIG. 12A is a photo of the Integra ASSIST PLUS pipetting robot 1200, with pipette holder 1201 on the left, tip box 1202, 96-well plate holder 1203, and custom chip adapter 1204. FIG. 12B depicts the tip box 1202 aligned under pipette holder 1201. FIG. 12C depicts the 96 well plate 1203 and adapter 1204 during functionalization.

TABLE 4
Integra ASSIST custom programs.

| | Program | Pipette Tip | 96 Well Palate | Application | Dispense Volume |
|---|---|---|---|---|---|
| # | Name | Location | Location | Volume (μL) | (μL)/Sensor Well |
| 1 | Dispense TE | A1, B1 | A1, B1 | 80 | 12 |
| 2 | Aspirate TE | I1, J1 | X | 12 | X |
| 3 | Dispense DSP | A2, B2 | A2, B2 | 80 | 12 |
| 4 | Aspirate DSP | I2, J2 | X | 12 | X |
| 5 | Dispense PNA | A, B(3-5); I, J(3-5) | A(3-8), B(3-8) | 15 | 12 |
| 6 | Aspirate PNA | A, B(6-8); I, J(6-8) | X | 12 | X |

First, the Tris-EDTA (TE) buffer is dispensed and removed from the chip surface to clean the surface and to ensure a tight seal of the micro-well array onto the sensing substrate. Then DSP, a bivalent cross-linking molecule, is introduced to the chip surface and readily adsorbs to the gold surface within 15-20 minutes. The presence of active NHS groups enables cross-linking to proteins (i.e., PNAs). Examples of linkers for attaching a capturing ligand/biological probe (such as PNA) are presented in Table 5. Finally, the DSP is aspirated and the PNA probes arc directly dispensed atop the sensing surface and couple to the free amines on the nanostructures. After the excess PNA solution is aspirated, the chip is covalently functionalized with PNAs and ready to use for sample testing.

TABLE 5
Attachment of Ligand to Gold.

| Active Group | |
|---|---|
| on Ligand | Cross-linking molecules for conjugation to gold |
| Amine | DSP (dithiobis(succinimidyl propionate)) |
| Cysteine | Direct attachment of thiol group |
| Thiol | Direct attachment of thiol group |
| Amine | EDC (1-ethyl-3-(3- |
| | dimethylaminopropyl)carbodiimide hydrochloride) |
| | and NHS (N-hydroxysulfosuccinimide) |
| Amine | p-mercaptobenzoic acid (p-MBA) self-assembled |
| | monolayer followed by EDC and NHS |

While pipetting robot systems have been widely utilized for fluid loading applications, to the best of our knowledge this is the first time such a system has been employed for covalently attaching a molecular capture probe to a solid-state sensor. We accomplish this through successive dispense and aspiration steps atop the sensors.

### Example 9: Plasmonic Sensor for HPV Screening

A plasmonic sensor can be used for amplification-free HPV genotyping and stratification of high-risk strains directly from processed cervical swab samples. The principle of operation relies on localized surface plasmon resonance (LSPR). LSPR employs a unique characteristic of metal nanoparticles. Particles collectively oscillate when excited by incident light, and this collective oscillation is highly sensitive to bulk and localized changes in refractive index. This plasmonic phenomenon results in a resonant peak wavelength of the nanosensor, which shifts upon a refractive index change near the sensing substrate. Herein, the nanoparticle array is covalently functionalized with peptide nucleic acid (PNA) probes complementary to at least a portion of target DNAs. The attached probes selectively bind target HPV DNAs, enabling highly sensitive and quantitative transduction following target DNA hybridization to PNA probes and sensing substrate. Together, these data present a streamlined method for functionalization and testing of plasmonic nanoarray substrates towards DNA detection.

All sensing experiments conducted for HPV employed a 2×6 array of 1 mm² nanosensors (12 sensors total). Each of the sensors consisted of 145 nm gold nanocubes with regular spacing. The selected nanoarray substrate contained twelve 1 mm² nanosensor arrays. In order to individually functionalize the nanosensor arrays to be target-specific, a polydimethylsiloxane (PDMS) polymer micro-well array was fabricated. This micro-well array was aligned with the substrate such that each nanosensor could be accessed through a single well. This approach created repeatable, programmable coordinates for the Integra ASSIST PLUS pipetting robot.

A mold for casting the PDMS micro-wells was designed in Solidworks consisting of twelve 2 mm×2 mm×5 mm (20 mm³) pillars. The pillars were positioned to match the coordinates of nanosensor array on the glass substrate. Master molds were made using SLA 3D printing. Micro-well array devices were fabricated in the molds using PDMS soft lithography. Sylgard 184 silicone elastomer, base and curing agent (Dow Corning, Midland, MI) were mixed in a ratio of 10:1, by weight. Next, the PDMS prepolymer was cast on the master mold and cured at 80° C. in a convection oven for approximately 1.5 h. The cured PDMS micro-well array was removed from the master mold and affixed to the nanosensor substrate using a washable glue stick, which allowed for removable bonding.

To covalently functionalize the nanosensor with selected PNA probes. the gold nanostructures on a glass substrate were incubated with 1 mg/mL dithiobis succinimidyl propionate (DSP) dissolved in dimethyl sulfoxide (DMSO) for 20 min. This crosslinking molecule activated the gold surface to enable coupling of free amines on the PNA. Next, the sensor arrays were put in contact with 1 mg/mL PNA probe dispersed in Tris-EDTA buffer (pH 7.0) for 30-45 min. Transmission spectra were collected before and after conjugation to characterize successful PNA conjugation.

The nanosensor functionalization process described above was automated using an Integra ASSIST PLUS pipetting robot. In order to effectively position the device onto the deck of the Integra ASSIST PLUS pipetting robot, we designed and fabricated a custom 4-slot microscope slide holder the size of a standard 96-well plate. This adapter was designed and 3D printed as described herein, and could readily be integrated with the liquid handler's robotic deck. A 96-well plate was pre-loaded with functionalization reagents and placed in the robot's aspiration deck. To start up the machine, a Voyager electronic 125 μL, 8-channel pipette was loaded onto the robot. A suite of six programs were developed to aspirate, dispense, and clear tips in an automated fashion.

### Example 10: Development of HPV Probes

In silico methods were used in designing PNA probes for nanoplasmonic characterization of the human papillomavirus (HPV) and related HPV infections in a subject. Two serotype-specific PNA probes for HPV16 and HPV18 as well as 5 additional probes that are collectively inclusive of all 14 hrHPV serotypes (HPV types 16, 18, 31, 33, 35, 39, 45, 51, 52, 56, 58, 59, 66, 68) were designed.

Genes that are conserved within the desired group of targeted pathogens, but distinct enough from their nearest neighbors were determined through literature survey and visual examination of the alignments for species identification. Reference target genes were subjected to BLAST against 5000 records in the nucleotide database to generate XML files containing complete results of alignments of homologous sequences (coverage/identity >80%). The XML files containing the alignment records were parsed into python using Biopython modules. Identical sequence records were grouped indicating the number of repeats and parsed into fasta files. Fasta files were used to realign the sequence records for further analysis.

The sequence alignments were visually examined to identify potential locations for probe placement. PNA probes were designed such that the Tₘ of the PNA-DNA duplex is ˜80 C. The probe lengths were kept <25 nt. The Tm of the PNA-DNA hybrid was determined using the PNA Bio Tool. Purine composition was kept <50% to avoid precipitation of PNA probes. Sequences that produce stable homodimers, and hairpins (Tm>30 C) were avoided.

Once the probe sequence was determined, the analytical inclusivity of the given probe was evaluated using multiple databases. All probes were tested against the NCBI's nucleotide database to retrieve a complete record of high-scoring pairs (HSPs). Parameters including Accession Number, Identity, Coverage, Number of mismatches, mismatched based and location, was retrieved using custom scripts. Identical results were grouped marking a single representative record and the number of records that duplicates the parameters. Additionally, based on the target. additional databases were used to further validate inclusivity/cross-reactivity using the same analysis criteria. HPV targets were tested against the HPV representative genome database to determine serotype inclusivity and cross-reactivity. HPV probes were also evaluated against the prokaryotic representative genome database to ensure the absence of cross-reactivity with prokaryotic pathogens.

A serotype-specific PNA probe for HPV16 was designed. A novel set of PNA probes including one that is highly specific to HPV18 as well as 5 additional probes that are collectively inclusive of all 14 hrHPV serotypes (HPV types 16, 18, 31, 33, 35, 39, 45, 51, 52, 56, 58, 59, 66, 68) were designed, as shown in Table 5.

TABLE 5
Summary of HPV Probes

| | SEQ | | |
|---|---|---|---|
| | ID | | |
| Probe | NO: | Sequence (5′-3′) | Gene |
| HPV16 | 1 | TTAGGCAGCACTTGGCCAACCA | E2 |
| HPV18 | 2 | AAACAGCTACAGCACACCCCCTCAC | E2 |
| HPV consensus | 3 | ACCTGACCACCCGCATGTACTTCCC | E2 |
| 1 | | | |
| HPV consensus | 4 | TGTATTATAGGCGTAGTGTTACCAC | E2 |
| 2 | | | |
| HPV consensus | 5 | CCGCTGTCCGGATAACTGTCCAG | E1 |
| 3 | | | |
| HPV consensus | 6 | AGTCCATAGCGCCATATCCTGTGTC | L1 |
| 4 | | | |
| HPV consensus | 7 | GTATTGCCATATCCGCTGTCTTCTG | E1 |
| 5 | | | |

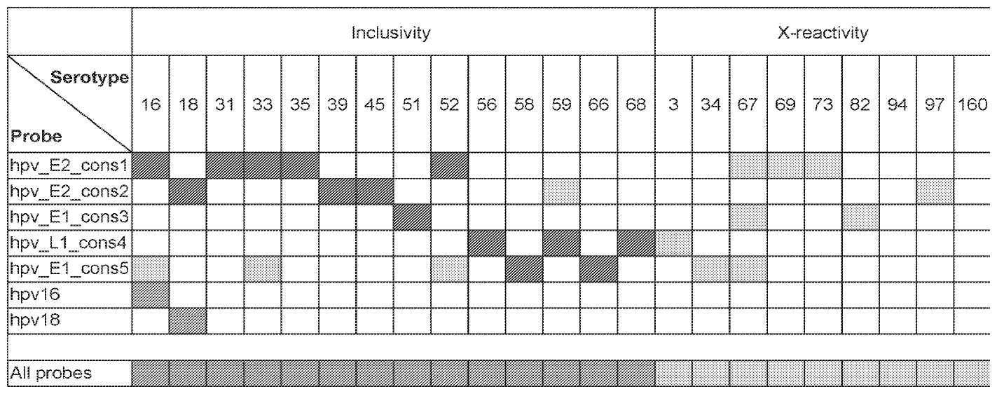

Due to the high heterogeneity between HPV serotypes, additional in silico analyses were carried out to minimize thermodynamic penalties due to any mismatches that may exist. All probe sequences were subjected to in silico analyses against representative HPV genomes available in the Papillomavirus Episteme (PaVE of NIAID) database to determine specificity and cross-reactivity (FIG. 13). The probes were also evaluated against sequence records available in the nucleotide database as well as with representative genomes of prokaryotes to confirm inclusivity and rule out cross-reactivity with other non-specific pathogens.

TABLE 6
Inclusivity/Cross-reactivity of HPV probes as determined with HPV
representative genomes

| | | | | |
|---|---|---|---|---|
| Serotype | Genbank ID | Mismatches | Mismatched base | Mismatched Loc |

HPV16 probe

| | | |
|---|---|---|
| HPV16 | K02718 | 0 |

HPV 18 probe

| | | |
|---|---|---|
| HPV18 | X05015 | 0 |

HPV consensus probe 1

| | | | | |
|---|---|---|---|---|
| HPV16 | K02718 | 1 | T-T | 18 |
| HPV31 | J04353 | 1 | T-G | 18 |
| HPV33 | M12732 | 1 | G-T | 13 |
| HPV35 | X74477 | 2 | G-T, T-G | 13, 18 |
| HPV52 | X74481 | 1 | G-T | 13 |
| HPV67 | D21208 | 3 | C-C, G-T, T-G | 7, 13, 18 |
| HPV69 | AB027020 | 3 | C-T, G-T, T-T | 11, 13, 21 |
| HPV73 | X94165 | 2 | G-T, C-A | 13, 14 |

HPV consensus probe 2

| | | | | |
|---|---|---|---|---|
| X05015 | HPV18 | 0 | | |
| M62849 | HPV39 | 0 | | |
| X74479 | HPV45 | 0 | | |
| X77858 | HPV59 | 3 | | |
| DQ080080 | HPV97 | 0 | | |
| MH777368 | HPV-mSK229 | 3 | --G, G-T, T-G | 9, 14, 21 |

HPV consensus probe 3

| | | | | |
|---|---|---|---|---|
| HPV51 | M62877 | 0 | | |
| HPV69 | AB027020 | 3 | T-T, A-G | 13, 14 |
| HPV82 | AB027021 | 1 | G-T | 3 |

HPV consensus probe 4

| | | | | |
|---|---|---|---|---|
| HPV3 | X74462 | 3 | A-C, G-T, T-T | 8, 11, 17 |
| HPV56 | X74483 | 1 | T-T | 15 |
| HPV59 | X77858 | 1 | G-G | 11 |
| HPV68 | DQ080079 | 2 | G-T, G-T | 11, 23 |
| HPV94 | AJ620211 | 3 | G-T, T-C, T-T | 11, 17, 20 |
| HPV160 | AB745694 | 3 | G-T, T-T, G-T | 11, 20, 23 |

HPV consensus probe 5

| | | | | |
|---|---|---|---|---|
| HPV16 | K02718 | 3 | T-G | 12 |
| HPV33 | M12732 | 1 | | |
| HPV34 | X74476 | 3 | | |
| HPV52 | X74481 | 3 | T-C | 11 |
| HPV58 | D90400 | 1 | | |
| HPV66 | U31794 | 1 | T-G | 12 |
| HPV67 | D21208 | 1 | | |

TABLE 7
Inclusivity/Cross-reactivity of HPV probes as determined with nucleotide
database. The results are sorted based on the number of counts and only the first 6 records per
probe is shown here.

| | | | | | | |
|---|---|---|---|---|---|---|
| Title | Accession | mismatches | Ident | Cov | Strand | count |

HPVE 16 probe

| | | | | | | | |
|---|---|---|---|---|---|---|---|
| 0 | Human papillomavirus type 16 | LC511112.1 | 0 | 100% | 100% | 1.00 | 3572 |
| 1 | Human papillomavirus type 16 | LC456618.1 | 1 | 95% | 95% | 1.00 | 239 |
| 2 | PREDICTED: Molossus molossus zinc | XM_036272575.1 | 1 | 95% | 100% | −1.00 | 33 |
| 3 | Human papillomavirus isolate HPV16 | KY502013.1 | 0 | 100% | 100% | 1.00 | 21 |
| 4 | Human papillomavirus type 16 | MG850728.1 | 1 | 95% | 100% | 1.00 | 18 |
| 5 | PREDICTED: Choloepus didactylus forkhead | XM_037832101.1 | 6 | 73% | 73% | 1.00 | 10 |

HPVE 18 probe

| | | | | | | | |
|---|---|---|---|---|---|---|---|
| 0 | Human papillomavirus type 18 | LC509006.1 | 0 | 100% | 100% | 1 | 121 |
| 1 | Eilema sororculum genome assembly, | OU618555.1 | 5 | 80% | 84% | 1 | 18 |
| 2 | Human papillomavirus isolate HPV18 | KY502032.1 | 0 | 100% | 100% | 1 | 16 |
| 3 | PREDICTED: Astyanax mexicanus DAZ | XM_022669714.1 | 6 | 76% | 76% | 1 | 9 |
| 4 | Human papillomavirus type 18 | LC540538.1 | 4 | 84% | 84% | 1 | 8 |
| 5 | PREDICTED: Cheilinus undulatus nuclear | XM_041806790.1 | 7 | 72% | 72% | 1 | 7 |

HPV consensus probe 1 (hpv_E2_cons1)

| | | | | | | | |
|---|---|---|---|---|---|---|---|
| 0 | Human papillomavirus type 16 | LC511112.1 | 1 | 96% | 100% | −1 | 3436 |
| 1 | Human papillomavirus type 31 | LR862018.1 | 1 | 96% | 100% | −1 | 1260 |
| 2 | Human papillomavirus 52 isolate | MZ374448.1 | 1 | 96% | 100% | −1 | 84 |
| 3 | Human papillomavirus type 52 | LC373207.1 | 1 | 96% | 100% | −1 | 56 |
| 4 | Human papillomavirus isolate HPV16 | KY501994.1 | 1 | 96% | 100% | −1 | 18 |
| 5 | Human papillomavirus type 33 | KU298894.1 | 1 | 96% | 100% | −1 | 10 |

HPV consensus probe 2 (hpv_E2_cons2)

| | | | | | | | |
|---|---|---|---|---|---|---|---|
| 0 | Human papillomavirus type 18 | LC509006.1 | 0 | 100% | 100% | −1 | 135 |
| 1 | Human papillomavirus type 39 | KX514417.1 | 0 | 100% | 100% | −1 | 28 |
| 2 | Human papillomavirus type 45 | KC470260.1 | 0 | 100% | 100% | −1 | 27 |
| 3 | Human papillomavirus type 59 | KU298921.1 | 3 | 88% | 88% | −1 | 26 |
| 4 | Hypena proboscidalis genome assembly, | LR990152.1 | 6 | 76% | 80% | −1 | 13 |
| 5 | Human papillomavirus isolate HPV18 | KY502036.1 | 7 | 72% | 72% | −1 | 12 |

HPV consensus probe 3 (hpv_E1_cons3)

| | | | | | | | |
|---|---|---|---|---|---|---|---|
| 0 | Human papillomavirus type 51 | KX514432.1 | 0 | 100% | 100% | −1 | 49 |
| 1 | Human papillomavirus type 82 | KX514421.1 | 3 | 87% | 87% | −1 | 23 |
| 2 | PREDICTED: Varroa destructor | XM_022817696.1 | 6 | 74% | 74% | 1 | 7 |
| | pyridoxine/pyridoxamine | | | | | | |
| 3 | PREDICTED: Varroa jacobsoni | XM_022842608.1 | 6 | 74% | 74% | 1 | 4 |
| | pyridoxine/pyridoxamine | | | | | | |
| 4 | PREDICTED: Bradysia coprophila | XM_037189567.1 | 4 | 83% | 87% | −1 | 4 |
| | microtubule-associated | | | | | | |
| 5 | PREDICTED: Chelmon rostratus KIAA0586 | XM_041954393.1 | 4 | 83% | 87% | 1 | 4 |

HPV consensus probe 4 (hpv_L1_cons4)

| | | | | | | | |
|---|---|---|---|---|---|---|---|
| 0 | Human papillomavirus type 59 | MK648147.1 | 1 | 96% | 100% | −1 | 88 |
| 1 | Human papillomavirus type 56 | KX645780.1 | 1 | 96% | 100% | −1 | 37 |
| 2 | Human papillomavirus type 68 | KX514431.1 | 2 | 84% | 88% | −1 | 26 |
| 3 | Bos taurus genome assembly, | LR962868.1 | 6 | 76% | 80% | 1 | 4 |
| 4 | Human papillomavirus type 56 | EF177181.1 | 2 | 92% | 100% | −1 | 2 |
| 5 | Human papillomavirus type 68a | LR861886.1 | 4 | 84% | 88% | −1 | 2 |

HPV consensus probe 5 (hpv_E1_cons5)

| | | | | | | | |
|---|---|---|---|---|---|---|---|
| 0 | Human papillomavirus type 16 | MG849855.1 | 3 | 88% | 92% | −1 | 3726 |
| 1 | Human papillomavirus type 31 | LR862018.1 | 5 | 80% | 84% | −1 | 366 |
| 2 | Human papillomavirus type 58 | LC376008.1 | 1 | 96% | 96% | −1 | 170 |
| 3 | Human papillomavirus 52 isolate | MZ374448.1 | 3 | 88% | 92% | −1 | 86 |
| 4 | Human papillomavirus type 52 | LC373207.1 | 3 | 88% | 92% | −1 | 82 |
| 5 | Human papillomavirus type 51 | KX514432.1 | 5 | 80% | 80% | −1 | 49 |

TABLE 8
Cross-reactivity of HPV probes against representative bacterial genomes

| Probe | Species | Accession | Isolate | % Identity | Mismatches | Count |
|---|---|---|---|---|---|---|
| hpv16 | Candidatus | NZ_CP016782 | Candidatus Planktophila | 91 | 2 | 1 |
| | Planktophila | | limnetica, complete | | | |
| | | | genome. | | | |
| hpv16 | Marinobacter | NZ_SWKM01000012 | Marinobacter alexandrii | 86 | 3 | 1 |
| | alexandrii | | strain LZ-8 NODE_2, | | | |
| | | | whole genome shotgun | | | |
| | | | sequence | | | |
| hpv18 | Metallibacterium | NZ_LDOS01000005 | Metallibacterium | 84 | 4 | 1 |
| | scheffleri | | scheffleri strain DKE6 | | | |
| | | | map unlocalized plasmid | | | |
| | | | unnamed31 p_31, whole | | | |
| | | | genome shotgun | | | |
| | | | sequence | | | |
| hpv18 | Paraburkholderia | NZ_VOQS01000005 | Paraburkholderia | 92 | 2 | 1 |
| | azotifigens | | azotifigens strain NF 2-5- | | | |
| | | | 3 4, whole genome | | | |
| | | | shotgun sequence | | | |
| hpv18 | Pseudomonas | NZ_BMQU01000012 | Pseudomonas | 76 | 6 | 1 |
| | laurentiana | | laurentiana strain JCM | | | |
| | | | 32154 sequence012, | | | |
| | | | whole genome shotgun | | | |
| | | | sequence | | | |
| hpv_E1_cons5 | Megasphaera | NZ_CP009240 | Megasphaera elsdenii | 76 | 6 | 1 |
| | elsdenii | | 14-14 chromosome, | | | |
| | | | complete genome | | | |
| hpv_E1_cons5 | Sphingobacterium | NZ_SUKA01000005 | Sphingobacterium | 76 | 6 | 1 |
| | alkalisoli | | alkalisoli strain Y3L14 | | | |
| | | | Scaffold5, whole | | | |
| | | | genome shotgun | | | |
| | | | sequence | | | |
| hpv_E1_cons5 | Thermoanaerobacter | NC_014209 | Thermoanaerobacter | 76 | 6 | 1 |
| | mathranii | | mathranii subsp. | | | |
| | | | mathranii str. A3, | | | |
| | | | complete sequence | | | |
| hpv_E1_cons5 | Virgibacillus | NZ_CCDP010000001 | Virgibacillus massiliensis | 84 | 4 | 1 |
| | massiliensis | | strain Vm-5, whole | | | |
| | | | genome shotgun | | | |
| | | | sequence | | | |
| hpv_E1_cons5 | Virgibacillus | NZ_BNEO01000002 | Virgibacillus salexigens | 84 | 4 | 1 |
| | salexigens | | strain DSM 11483 | | | |
| | | | NODE_2, whole genome | | | |
| | | | shotgun sequence | | | |
| hpv_E2_cons1 | Albibacillus | NZ_QEHM01000007 | Albibacillus kandeliae | 84 | 4 | 1 |
| | kandeliae | | strain J95 Scaffold7, | | | |
| | | | whole genome shotgun | | | |
| | | | sequence | | | |
| hpv_E2_cons1 | Bordetella | NZ_UFUJ01000001 | Bordetella trematum | 88 | 3 | 1 |
| | trematum | | strain NCTC12995, | | | |
| | | | whole genome shotgun | | | |
| | | | sequence | | | |
| hpv_E2_cons1 | Gordonia | NZ_BAHE01000020 | Gordonia namibiensis | 88 | 3 | 1 |
| | namibiensis | | NBRC 108229, whole | | | |
| | | | genome shotgun | | | |
| | | | sequence | | | |
| hpv_E2_cons1 | Leifsonia | NZ_JACCFM010000001 | Leifsonia | 80 | 5 | 1 |
| | psychrotolerans | | psychrotolerans strain | | | |
| | | | LI1 Ga0415210_01, | | | |
| | | | whole genome shotgun | | | |
| | | | sequence | | | |
| hpv_E2_cons1 | Mesorhizobium | NZ_QMBQ01000001 | Mesorhizobium | 96 | 1 | 1 |
| | atlanticum | | atlanticum strain CNPSo | | | |
| | | | 3140 scaffold_0, whole | | | |
| | | | genome shotgun | | | |
| | | | sequence | | | |
| hpv_E2_cons1 | Noviherbaspirillum | NZ_JAEPBG010000016 | Noviherbaspirillum | 84 | 4 | 1 |
| | pedocola | | pedocola strain DKR-6 | | | |
| | | | contig 16, whole genome | | | |
| | | | shotgun sequence | | | |
| hpv_E2_cons1 | Paenibacillus | NZ_JAELUP010000031 | Paenibacillus roseus | 84 | 4 | 1 |
| | roseus | | strain MAHUQ-46 | | | |
| | | | velvet_auto_83_31, | | | |
| | | | whole genome shotgun | | | |
| | | | sequence | | | |
| hpv_E2_cons1 | Pontibacter | NZ_CP021235 | Pontibacter actiniarum | 88 | 3 | 1 |
| | actiniarum | | strain DSM 19842 | | | |
| | | | chromosome, complete | | | |
| | | | genome | | | |
| hpv_E2_cons1 | Pontibacter | NZ_CP047984 | Pontibacter russatus | 88 | 3 | 1 |
| | russatus | | strain BT326 | | | |
| | | | chromosome, complete | | | |
| | | | genome | | | |
| hpv_E2_cons1 | Rhodococcus | NZ_LRRH01000037 | Rhodococcus phenolicus | 88 | 3 | 1 |
| | phenolicus | | strain DSM 44812 | | | |
| | | | contig_38, whole | | | |
| | | | genome shotgun | | | |
| | | | sequence | | | |
| hpv_E2_cons1 | Roseomonas | NZ_CP015583 | Roseomonas gilardii | 84 | 4 | 1 |
| | gilardii | | strain U14-5 | | | |
| | | | chromosome 1, | | | |
| | | | complete sequence. | | | |
| hpv_E2_cons1 | Saccharothrix | NZ_JAGINVO10000001 | Saccharothrix | 88 | 3 | 1 |
| | coeruleofusca | | coeruleofusca strain | | | |
| | | | DSM 43679 | | | |
| | | | Ga0451103_01, whole | | | |
| | | | genome shotgun | | | |
| | | | sequence | | | |

### Example 11: In Vitro HPV Genotyping using Nanoplasmonic Sensors

The sensor was functionalized with PNA sequences selected from Table 9. Specifically. both a HPV16-specific (SEQ ID NO: 1) and a HPV consensus (SEQ ID NO: 8) PNA were used, in addition to a negative control PNA.

TABLE 9
Selected PNA Probe Sequences for HPV16 and
Consensus hrHPV

| Name | SEQ ID NO: | Sequence |
|---|---|---|
| HPV16 | 1 | TTAGGCAGCACTTGGCCAACCA |
| HPV Consensus | 8 | CGTCCCAAAGGAAACTGATC |

The functionalized sensor was tested with known high concentrations (10,000-100,000 copies/mL) of synthetic complementary oligos. Large, measurable peak shifts (>3 nm) were observed, demonstrating sensor readiness for testing patient samples with unknown concentration.

### Example 12: Methodology for Sample Preparation and Processing of Clinical Samples

50 discarded, deidentified patient samples were collected from the Center for Clinical Genomics and Advanced Technology at Dartmouth-Hitchcock Medical Center. All samples were ThinPrep cervical swab samples that had undergone nucleic acid extraction procedures. Samples were collected from the Dartmouth-Hitchcock catchment region, as well as through outreach work with the Center for Global Oncology at Norris Cotton Cancer Center at Dartmouth-Hitchcock Medical Center. In total, the cervical swab samples were collected from New Hampshire, Kosovo, and Honduras. Nucleic acid extraction was either performed on the automated EZ1 bacterial card/tissue or using the Atila Nucleic Acid Extraction Kit. All gold-standard genotyping was performed using the Roche Cobas HPV test. This commercially available test can differentiate HPV16, HPV18, and other pooled hrHPVs (31, 33, 35, 39, 45, 51, 52, 56, 58, 59, 66, and 68). In the case where the sample was processed as “other hrHPV”. it reflexed to the Atila Multiplex High-Risk HPV test for genotyping. Total nucleic acid concentration (human and HPV) of the extracted samples was provided to the Nanopath team. and ranged from 0.56-74.8 ng/uL. All samples were deidentified, and the team was blinded to the results until after sample processing and data analysis.

The 50 discarded, deidentified samples were stored at −80° C. Prior to sample analysis, the microwell array was affixed to the nanosensor array, and the combined array affixed to a 75×25 mm microchip. A transmittance measurement was taken through the dry chip to confirm appropriate alignment and signal from each sensing spot. The nanosensing spots were then functionalized. Each sample was exposed to a negative control probe, a HPV16 probe, and a HPV consensus probe. A fully functionalized chip with twelve nanosensing arrays was used to test a total of four patient samples. The samples were thawed and 8 uL was pipetted into each sensing spot. A transmittance spectrum was collected as soon as the sample was delivered and again after a 5-minute incubation.

All transmittance spectra were collected using Nanopath's custom built readout instrumentation coupled to our integrated user interface. The assembly comprises a linear stage, a light source, a spectrometer, and focused lens components. The readout instrumentation moves the slide to designated coordinate locations and measures the transmittance from 500 nm-1000 nm wavelengths through the sample. For each sample, paired measurements are taken through the nanoarray and through a background location. This data is then analyzed within the integrated user interface.

The normalized transmittance spectrum was calculated as the ratio of the signal to background at every wavelength. The extinction was then calculated as the negative natural log of the normalized transmittance. These extinction spectra were smoothed using Lowess smoothing in MATLAB (10% smoothing) before the resonance peak wavelength was calculated. The resonance peak wavelength was determined through a center of mass calculation using numerical integration with wavelength bounds 750 nm to 975 nm. Spectral shifts were calculated by subtracting sample resonance peak locations before and after sample incubation. The UI returns a positive/negative for each sample, defined as a spectral shift ≥1 nm.

### Example 13: HPV Genotyping of Clinical Samples using Nanoplasmonic Sensors

Using the two probes of SEQ ID NO: 1 and SEQ ID NO 8, 50 processed ThinPrep cervical swab samples were analyzed using a sensor, reader, and UI. The experiments were conducted as a blind study to the genotype of each sample until after sample processing and analysis. Each 12-plex sensor chip was functionalized for processing 4 patient samples each on the 3 selected probes (HPV16, HPV consensus, and negative control). All samples were processed as described above and in Tables 10 and 11, and a peak shift of greater than or equal to 1 nm was defined as a positive result.

TABLE 10

| Sample breakdown, including DNA extraction method and total DNA | |
|---|---|
| concentrations | |
| Extraction Method | # Samples (total = 50) |
| EZ1 tissue kit | 28 |
| Dneasy tissue kit | 3 |
| Atila | 19 |

TABLE 11
Sensitivity and specificity results

| Total DNA | |
|---|---|
| concentration (ng/ul) | # Samples (total = 50) |
| <5 ng/uL | 8 |
| 5-20 ng/uL | 29 |
| >20 ng/uL | 13 |

In spite of the variability in sample handling & total DNA concentration, the results demonstrated exceptional sensitivity and specificity. In particular, the HPV16 probe conferred in 100% (32/32) specificity, and 93% (14/15) sensitivity for HPV detection in a sample. The other high-risk HPV probe conferred 100% (34/34) specificity, and 92% (12/13) sensitivity for HPV detection in a sample. There were no false positives identified in this testing (i.e. 100% specificity), and sensitivity exceeded 92%. A comprehensive breakdown of patient samples, genotypes, and raw results are as found in Tables 12 and 13.

TABLE 12
Patient-level data for patients #1-25. Infection is determined through gold-
standard Roche Cobas or Atila genotyping. Results were determined through Nanopath
processing. Quantified peak shifts were measured with standard error of the mean for each
sample.

| Sample ID | Infection | Result | HPV 16 | Consensus | Negative Control |
|---|---|---|---|---|---|
| TP-01 | HPV-31 | Consensus Primer | 0.34 ± 0.2 | 2.21 ± 0.2 | 0.09 ± 0.1 |
| TP-02 | HPV-59, HPV-66, HPV-31 | Consensus Primer | −0.12 ± 0.06 | 1.26 ± 0.5 | −0.09 ± 0.1 |
| TP-03 | HPV-45 | Consensus Primer | −0.45 ± 0.2 | 1.65 ± 0.03 | −0.13 ± 0.1 |
| TP-04 | HPV-66 | None | 0.48 ± 0.5 | 0.14 ± 0.1 | −0.07 ± 0.2 |
| TP-05 | HPV-51, HPV-56 | Moderate Consensus | 0.21 ± 0.23 | 0.52 ± 0.06 | −0.33 ± 0.06 |
| | | Binding | | | |
| TP-06 | HPV-31 | Consensus Primer | −0.05 ± 0.41 | 1.41 ± 0.02 | 0.01 ± 0.27 |
| TP-07 | HPV-35 | Consensus Primer | −0.41 ± 0.5 | 2.07 ± 0.4 | 0.03 ± 0.1 |
| TP-08 | HPV-56 | None | −0.17 ± 0.2 | .17 ± 0.3 | −0.12 ± 0.3 |
| TP-09 | HPV-66 | None | −0.18 ± 0.6 | 0.67 ± 0.8 | 0.32 ± 0.4 |
| TP-10 | HPV-59 | None | 0.29 ± 0.22 | 0.29 ± 0.23 | 0.06 ± 0.27 |
| TP-11 | HPV-56 | None | 0.10 ± 0.5 | 0.2 ± 0.6 | −0.05 ± 0.6 |
| TP-12 | HPV-31 | Consensus Primer | 0.3 ± 0.2 | 1.51 ± 0.7 | −0.04 ± 0.2 |
| TP-13 | HPV-52 | None | −0.14 ± 0.2 | 0.09 ± 0.05 | 0.00 ± 0.4 |
| TP-14 | HPV-39 | None | −0.18 ± 0.31 | 0.20 ± 0.03 | −0.01 ± 0.29 |
| TP-15 | HPV-58 | Consensus Primer | −0.03 ± 0.06 | 1.33 ± 0.01 | 0.14 ± 0.04 |
| TP-16 | Negative | None | 0.02 ± 0.2 | 0.24 ± 0.2 | 0.09 ± 0.5 |
| TP-17 | Negative | None | 0.13 ± 0.02 | 0.13 ± 0.00 | 0.15 ± 0.12 |
| TP-18 | Negative | None | 0.23 ± 0.11 | 0.32 ± 0.11 | 0.16 ± 0.10 |
| TP-19 | Negative | None | 0.17 ± 0.10 | 0.33 ± 0.09 | −0.39 ± 0.08 |
| TP-20 | Negative | None | −0.16 ± 0.54 | 0.04 ± 0.55 | −0.36 ± 0.24 |
| TP-21 | HPV-16 | 16 Primer, Consensus | 1.86 ± 0.2 | 1.58 ± 0.2 | −0.03 ± 0.3 |
| | | Primer | | | |
| TP-22 | HPV-16 | 16 Primer, Consensus | 1.82 ± 0.2 | 1.19 ± 0.6 | 0.09 ± 0.6 |
| | | Primer | | | |
| TP-23 | HPV-16 | 16 Primer, Consensus | 2.97 ± 0.5 | 1.49 ± 0.5 | 0.27 ± 0.2 |
| | | Primer | | | |
| TP-24 | HPV-16 | 16 Primer, Consensus | 1.43 ± 0.1 | 0.95 ± 0.03 | −0.40 ± 0.02 |
| | | Primer | | | |
| TP-25 | HPV-16 | 16 Primer, No | 1.91 ± 0.003 | 0.32 ± 0.4 | −0.13 ± 0.3 |
| | | Consensus Primer | | | |

TABLE 13
Patient-level data for patients #26-50. Infection is determined through gold-
standard Roche Cobas or Atila genotyping. Results were determined through Nanopath
processing. Quantified peak shifts were measured with standard error of the mean for each
sample.

| Sample ID | Infection | Result | HPV 16 | Consensus | Negative Control |
|---|---|---|---|---|---|
| TP-26 | Negative | None | 0.03 ± 0.33 | 0.27 ± 0.19 | −0.27 ± 0.67 |
| TP-27 | Negative | None | 0.11 ± 0.4 | −0.08 ± 0.3 | 0.1 ± 0.2 |
| TP-28 | Negative | None | −0.21 ± 0.26 | −0.14 ± 0.33 | 0.34 ± 0.21 |
| TP-29 | Negative | None | 0.11 ± 0.4 | −0.09 ± 0.02 | −0.17 ± 0.02 |
| TP-30 | Negative | None | 0.12 ± 0.59 | 0.06 ± 0.28 | 0.60 ± 0.17 |
| TP-31 | Negative | Bound negative control | 0.04 ± 0.06 | −0.16 ± 0.02 | 0.95 ± 0.2 |
| TP-32 | Negative | None | 0.10 ± 0.03 | −0.13 ± 0.11 | 0.07 ± 0.23 |
| TP-33 | Negative | None | 0.39 ± 0.01 | 0.19 ± 0.04 | 0.4 ± 0.03 |
| TP-34 | Negative | None | −0.11 ± 0.06 | −0.38 ± 0.09 | 0.00 ± 0.04 |
| TP-35 | Negative | None | −0.14 ± 0.36 | −0.31 ± 0.39 | −0.34 ± 0.36 |
| TP-36 | HPV-16 | 16 Primer, Consensus | 1.78 ± 0.37 | 1.08 ± 0.42 | −0.38 ± 0.26 |
| | | Primer | | | |
| TP-37 | HPV-16, HPV 18 | 16 Primer, Consensus | 1.55 ± 0.2 | 1.37 ± 0.3 | 0.0 ± 0.1 |
| | | Primer | | | |
| TP-38 | HPV-16, HPV 18 | 16 Primer, Moderate | 1.98 ± 0.1 | 0.82 ± 0.04 | −0.2 ± 0.05 |
| | | Consensus Primer | | | |
| TP-39 | HPV-16, HPV 18 | 16 Primer, Consensus | 1.03 ± 0.05 | 1.52 ± 0.02 | 0.4 ± 0.03 |
| | | Primer | | | |
| TP-40 | HPV-16 | 16 Primer, Consensus | 1.26 ± 0.32 | 0.95 ± 0.05 | −0.11 ± 0.04 |
| | | Primer | | | |
| TP-41 | Negative | None | 0.08 ± 0.2 | 0.00 ± 0.4 | 0.31 ± 0.6 |
| TP-42 | HPV-16 | Consensus Only | −0.07 ± 0.04 | 0.96 ± 0.16 | −0.15 ± 0.05 |
| TP-43 | HPV-16 | 16 Primer, Consensus | 1.21 ± 0.06 | 1.51 ± 0.53 | 0.05 ± 0.03 |
| | | Primer | | | |
| TP-44 | HPV-16 | 16 Primer, Consensus | 1.90 ± 0.48 | 1.59 ± 0.08 | 0.35 ± 0.16 |
| | | Primer | | | |
| TP-45 | HPV-16, HPV-18 | 16 Primer, Consensus | 1.80 ± 0.04 | 1.05 ± 0.18 | −0.13 ± 0.06 |
| | | Primer | | | |
| TP-46 | HPV-16 | 16 Primer, Consensus | 1.72 ± 0.35 | 1.74 ± 0.15 | −0.28 ± 0.04 |
| | | Primer | | | |
| TP-47 | Negative | None | 0.06 ± 0.2 | −0.1 ± 0.1 | −0.4 ± 0.2 |
| TP-48 | Negative | None | 0.15 ± 0.12 | 0.40 ± 0.12 | 0.03 ± 0.14 |
| TP-49 | Negative | None | 0.05 ± 0.11 | −0.01 ± 0.11 | 0.04 ± 0.10 |
| TP-50 | Negative | None | 0.12 ± 0.2 | −0.32 ± 0.6 | 0.5 ± 0.3 |

The initial clinical sample testing above shows the potential to genotype individual high-risk HPV sequences (i.e. HPV16) as well as the ability to broadly identify a pool of hrHPVs. One challenge identified in the literature is the ability to broadly capture all 12+ high-risk HPVs through a small number of probes. Most existing approaches use up to twelve separate probes to ensure adequate coverage of all high-risk genotypes. Through careful probe design, a set of five consensus primers were designed with inclusivity over fourteen well-known high-risk genotypes (Table 5). These probes were also designed to be thermodynamically favorable PNA probes with low purine content and high melting temperatures. To the best of the Applicant's knowledge, these are among the best probes for consensus high-risk HPV genotyping due to their broad inclusivity. There is minimal cross-reactivity between these probes and other low-risk HPV genotypes (FIG. 13), thus reducing the risk of false negatives in hrHPV stratification.

The scope of the present disclosure is not intended to be limited by the specific disclosures of examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A nanoplasmonic sensor comprising:
an array of functionalized sensors;
wherein each of the functionalized sensors in the array comprises an array of nanostructures conjugated to a biological probe; and
the biological probe is configured to detect the presence of a human papillomavirus.

2. The nanoplasmonic sensor of claim 1, wherein the biological probe is a peptide nucleic acid probe or an oligonucleotide probe.

3. The nanoplasmonic sensor of claim 1, wherein at least one of the functionalized sensors in the array comprises a different biological probe for detecting a different segment or species of human papillomavirus from the other functionalized sensors.

4. The nanoplasmonic sensor of claim 3, wherein the nanoplasmonic sensor is configured to simultaneously detect multiple strains, segments, particles, mutants, and/or species of the human papillomaviruses.

5. The nanoplasmonic sensor of claim 3, wherein each of the functionalized sensors in the array comprises a different biological probe.

6. The nanoplasmonic sensor of claim 1, wherein the biological probe has a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

7. The nanoplasmonic sensor of claim 1, wherein the human papillomavirus is selected from the group consisting of HPV18, HPV16, hrHPV, HPV type 16, HPV type 18, HPV type 31, HPV type 33, HPV type 35, HPV type 39, HPV type 45, HPV type 51, HPV type 52, HPV type 56, HPV type 58, HPV type 59, HPV type 66, HPV type 68, and a derivative/mutant strain thereof.

8. The nanoplasmonic sensor of claim 1, wherein the nanostructures comprise gold.

9. The nanoplasmonic sensor of claim 1, wherein the nanostructures in the array are regularly-spaced apart with a spacing of from about 100 nm and about 2000 nm, and each nanostructure has a square shape with a side dimension of from about 50 nm to about 400 nm.

10. The nanoplasmonic sensor of claim 9, wherein the nanostructures have a thickness of from about 20 nm to about 75 nm.

11. The nanoplasmonic sensor of claim 1 wherein a single biological probe can bind nucleic acids derived from more than one high-risk HPV genotypes.

12. A method for detecting the presence of one or more human papillomaviruses, comprising:
exposing the nanoplasmonic sensor of claim 1 to a bodily fluid sample of a patient suspecting of having a human papillomavirus infection;
illuminating a light at a series of wavelengths onto each of the functionalized sensors; and
collecting absorbance, transmittance, or extinction data of each functionalized sensor.

13. The method of claim 12, further comprising heating the nanoplasmonic sensor after exposing the nanoplasmonic sensor to the bodily fluid sample.

14. The method of claim 12, where the body fluid sample is first exposed to a thermal, a mechanical, a chemical, or a biological treatment such that the human papillomavirus capsids are lysed before exposing to the nanoplasmonic sensor.

15. The method of claim 12, further comprises comparing the collected absorbance, transmittance, or extinction data of each functionalized sensor with a baseline data of each of the functionalized sensor prior to exposure to the bodily fluid sample.

16. The method of claim 15, wherein the comparing step reveals an optical peak shift when a human papillomavirus is detected.

17. The method of claim 16, wherein the amount of the optical peak shift is correlated to the concentration of the human papillomavirus in the bodily fluid sample.

18. The method of claim 12, wherein the bodily sample comprises mucus.

19. The method of claim 12, wherein at least one of the functionalized sensors in the array comprises a different biological probe for detecting a different human papillomavirus from the other functionalized sensors.

20. The method of claim 19, wherein the human papillomavirus is selected from the group consisting of HPV18, HPV16, hrHPV, HPV type 16, HPV type 18, HPV type 31, HPV type 33, HPV type 35, HPV type 39, HPV type 45, HPV type 51, HPV type 52, HPV type 56, HPV type 58, HPV type 59, HPV type 66, HPV type 68, and a derivative/mutant strain thereof.

21. The method of claim 19, wherein multiple strains, segments, particles, mutants, and/or species of the human papillomaviruses are detected simultaneously.

22. The method of claim 12, wherein the biological probe has a sequence independently selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

23. The method of claim 12, wherein each of the functionalized sensors in the array comprises a different biological probe.

24. The method of claim 12, wherein the method is configured to be performed at the point of care.

25. A method for detecting the presence of one or more human papillomaviruses, comprising:
providing a sensor comprising one or more biological probes designed to detect one or more target nucleic acid sequences derived from one or more human papillomaviruses;
exposing the sensor to a sample that is suspected to contain one or more human papillomaviruse; and
collecting electrical, fluorescent, absorbance, transmittance, and/or extinction data from the sensor.

26. The method of claim 25 wherein the one or more biological probes were selected using computational and/or bioinformatic methods.

27. The method of claim 25 wherein the one or more biological probes contain intentionally varying degrees of mismatch with the one or more target nucleic acid sequences.

28. The method of claim 25 wherein the one or more biological probes are designed to bind multiple target nucleic acid sequences.

29. The method of claim 25 wherein one of the biological probes can bind nucleic acids derived from more than one high-risk HPV genotypes.


SEQUENCE LISTING (8 sequences)

SEQ ID NO: 1 - DNA, 22 bp, Human papillomavirus 16
  source 1..22: mol_type = other DNA
ttaggcagcacttggccaacca

SEQ ID NO: 2 - DNA, 25 bp, Human papillomavirus 18
  source 1..25: mol_type = other DNA
aaacagctacagcacaccccctcac

SEQ ID NO: 3 - DNA, 25 bp, Human papillomavirus
  source 1..25: mol_type = other DNA
acctgaccacccgcatgtacttccc

SEQ ID NO: 4 - DNA, 25 bp, Human papillomavirus
  source 1..25: mol_type = other DNA
tgtattataggcgtagtgttaccac

SEQ ID NO: 5 - DNA, 23 bp, Human papillomavirus
  source 1..23: mol_type = other DNA
ccgctgtccggataactgtccag

SEQ ID NO: 6 - DNA, 25 bp, Human papillomavirus
  source 1..25: mol_type = other DNA
agtccatagcgccatatcctgtgtc

SEQ ID NO: 7 - DNA, 25 bp, Human papillomavirus
  source 1..25: mol_type = other DNA
gtattgccatatccgctgtcttctg

SEQ ID NO: 8 - DNA, 20 bp, Human papillomavirus
  source 1..20: mol_type = other DNA
cgtcccaaaggaaactgatc